(19) United States
(12) Patent Application Publication
(10) Pub. No.: US 2019/0108287 A1
(43) Pub. Date: Apr. 11, 2019

(54) MENU GENERATION SYSTEM TYING HEALTHCARE TO GROCERY SHOPPING

(71) Applicant: NutriStyle Inc, Seattle, WA (US)

(72) Inventors: Scott Murdoch, Bend, OR (US); Todd Albro, Eagle, ID (US); Caleb Skinner, Meridian, ID (US); Shannon Madsen, Meridian, ID (US)

(21) Appl. No.: 16/158,168

(22) Filed: Oct. 11, 2018

Related U.S. Application Data
(60) Provisional application No. 62/570,911, filed on Oct. 11, 2017.

(51) Int. Cl. G06F 17/30; G06Q 30/06; G16H 50/30

(52) U.S. Cl. CPC G06F 17/30867; G16H 50/30; G06Q 30/0633; G06Q 30/0641

(57) ABSTRACT

A system comprising a user interface and a controller operating to receive an input at a control interface. The system generates a control signal associated with the control interface. The system operates one or more filters to select a first set of food items from a proximal food distributor database and a menu rules control. The system generates a menu generation control signal utilizing the first set of food items selected and the menu rules control. The system sends the menu generation control signal to a menu generator and in response receive a menu control signal from the menu generator, the menu control signal associated with a second set of food items in the proximal food distributor database, wherein the second set of food items is a subset of the first set of food items. The system alters the machine display based on the menu control signal.

### CROSS-REFERENCE TO RELATED APPLICATIONS

This application claims priority and benefit under 35 U.S.C. 119 to U.S. application Ser. No. 62/570,911 filed on Oct. 11, 2017, titled “MENU GENERATION SYSTEM TYING HEALTHCARE TO GROCERY SHOPPING”.

### BACKGROUND

Lifestyle and health goals vary greatly from one person to the next. Conventional nutritional programs utilize various types of pre-existing menus that must either be modified by hand to properly meet the needs of any given person or used without change, resulting in an imperfect fit. As these programs do not fully consider the lifestyle and health goals of each individual, the program may be less likely to be adhered to, and therefore may not accomplish the goals of the individual even if adhered to. Conventional nutritional programs may also lack specificity as to which food items to consume, providing a more abstract food item, such as “low fat yogurt” instead of “Brand X low fat yogurt located in aisle N of Store Y located at address Z,” resulting in imprecise calculations of nutrient values for menus incorporating the foods.

### BRIEF SUMMARY

The present system and method utilizes user characteristics, health needs and preferences, and foods' actual characteristics as an input to nutritional algorithms to generate unique, alterable meal/snack and activity plans, menus, and shopping lists tied to the specific products available at a user's particular grocer. The present system and methods may utilize an existing food database (e.g., for a specific grocer) to provide a variety of personalized menu plans.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an embodiment of a system 100.

FIG. 2 illustrates an embodiment of a controller 200.

FIG. 3 illustrates an embodiment of a menu rules control 300.

FIG. 4 illustrates an embodiment of a user profile configuration UI 400.

FIG. 5 illustrates an embodiment of a menu preferences configuration UI 500.

FIG. 6 illustrates an embodiment of a daily menu UI 600.

FIG. 7 illustrates a filter alteration method 1700 in accordance with one embodiment.

FIG. 8 illustrates a filter alteration method 1700 in accordance with one embodiment.

FIG. 9 illustrates an embodiment of an item swap UI 900.

FIG. 10 illustrates a shopping list configuration UI 1000 in accordance with one embodiment.

FIG. 11 illustrates a shopping list configuration UI 1000 in accordance with one embodiment.

FIG. 12 illustrates a shopping list configuration UI 1000 in accordance with one embodiment.

FIG. 13 illustrates a food log configuration UI 1300 in accordance with one embodiment.

FIG. 14 illustrates an embodiment of a recipe creator UI 1400.

FIG. 15 illustrates a recipe creator UI 1400 in accordance with one embodiment.

FIG. 16 illustrates an embodiment of a meal plan generation process 1600.

FIG. 17 illustrates an embodiment of a filter alteration method 1700.

FIG. 18 illustrates an embodiment of a menu generator process 1800.

FIG. 19 illustrates a method 1900 in accordance with one embodiment.

FIG. 20 illustrates a system 2000 in accordance with one embodiment.

### DETAILED DESCRIPTION

“Comparator” refers to a logic element that compares two or more inputs to produce one or more outputs that reflects similarity or difference of the inputs. An example of a hardware comparator is an operational amplifier that outputs a signal indicating whether one input is greater, less than, or about equal to the other. An example software or firmware comparator is: if (input1==input2) output=val1; else if (input1>input2) output=val2; else output=val3; Many other examples of comparators will be evident to those of skill in the art, without undo experimentation.

“One or more filters” refers to components utilized to alter a control signal.

“Correlator” refers to a logic element that identifies a configured association between its inputs. One examples of a correlator is a lookup table (LUT) configured in software or firmware. Correlators may be implemented as relational databases. An example LUT correlator is: |1ow_alarm_condition |low_threshold_value|0 | |safe_condition_|safe_lower_bound |safe_upper_bound| |high_alarm_condition|high_threshold_value| | Generally, a correlator receives two or more inputs and produces an output indicative of a mutual relationship or connection between the inputs. Examples of correlators that do not use LUTs include any of a broad class of statistical correlators that identify dependence between input variables, often the extent to which two input variables have a linear relationship with each other. One commonly used statistical correlator is one that computes Pearson's product-moment coefficient for two input variables (e.g., two digital or analog input signals). Other well-known correlators compute a distance correlation, Spearman's rank correlation, a randomized dependence correlation, and Kendall's rank correlation. Many other examples of correlators will be evident to those of skill in the art, without undo experimentation.

“Controller” refers to logic, collection of logic, or circuit that coordinates and controls the operation of one or more input/output devices and synchronizes the operation of such devices with the operation of the system as a whole. For example, the controller may operate as a component or a set of virtual storage processes that schedules or manages shared resources. For example, IF (controller.logic {device1|device2|device3} {get.data( ), process.data( ),store.data( )}), -device1 get.data(input1) -> data.input1; -device2 process.data(data.input1) -> formatted.data1 -> -device3 store.data(formatted.data1).

“Selector” refers to a logic element that selects one of two or more inputs to its output as determined by one or more selection controls. Examples of hardware selectors are multiplexers and demultiplexers. An example software or firmware selector is: if (selection_control==true) output = input1); else output = input2; Many other examples of selectors will be evident to those of skill in the art, without undo experimentation.

“Control signal” refers to instructions to operate a user interface, controller, etc.

“Classifier” refers to a specific type of correlator/associator logic that associates one or more inputs with a category, class, or other group sharing one or more common characteristics. An example of a classifier that may commonly be implemented in programmable hardware is a packet classifier used in network switches, firewalls, and routers (e.g., packet classifiers utilizing Ternary Content Addressable Memories). An example software or firmware classifier is: if (input1.value<12.5) input1.group = group1; else if (input1.value >= 12.5 and input1.value<98.1) input1.group = group2; else input1.group = group3; Other examples of classifiers will be readily apparent to those of skill in the art, without undo experimentation.

“Control interface” refers to a component of a user interface capable of transforming an input into a control signal to operate a controller.

“Food item” refers to a digital representation of a substance consumed to provide nutritional support for an organism, and may be represented as a combination of various nutrients, such as protein, carbohydrates, fats, calories, vitamins, etc. For example, food item may be an assortment of consumable substances that includes meats, grains, dairy products, fruits, mushrooms, and vegetables. The food items may include condiments such as spices that may be added in combination to the aforementioned food items. Furthermore, food items may include beverages. Individual food items may be combined as components of a meal.

“Food distributor portal” refers to an online store that displays merchandise, such as food items, and an order form.

“Combiner” refers to a logic element that combines two or more inputs into fewer (often a single) output. Example hardware combiners are arithmetic units (adders, multipliers, etc.), time-division multiplexers, and analog or digital modulators (these may also be implemented is software or firmware). Another type of combiner builds an association table or structure (e.g., a data structure instance having members set to the input values) in memory for its inputs. For example: val1, val2, val3->combiner logic->{val1, val2, val3} set.val1=val1; set.val2=val2; set.val3=val3; Other examples of combiners will be evident to those of skill in the art without undo experimentation.

“Food distributor” refers to any purveyor (e.g., grocery store, grocery delivery service, etc.) that primarily offers ingredients to a user to utilize as the components of a meal, with the unit size of the ingredient being greater than the quantity required for an individual meal portion. A main difference between a food distributor and a restaurant/food service is in the quantity of the components usually exceeding the quantity required for a single meal.

“Proximal food distributor database” refers to an accessible database of food items associated with a food distributor that is any purveyor (e.g., grocery store, grocery delivery service, etc.) where the geographic location of the food distributors physical food items are a factor in their proximity to the user.

A method of operating a menu generation system tying healthcare and grocery shopping involves storing user profile information inputs, an input for a proximal food distributor database, and one or more menu influencer parameters from a user profile configuration user interface (UI) and menu rules controls, a menu filter, brand influencers, and food item influencers from a menu preferences configuration UI as a user profile in a user profile control memory data structure. The brand influencers and the food item influencers each comprise an influencer type. The system filters one or more food items from the proximal food distributor database to generate a first set of food items through operation of one or more filters configured by the food item influencers and brand influencers. The system generates a second set of food items from the first set of food items through operation of a menu generator configured by the one or more menu influencer parameters, menu rules control, and the menu filter. The system displays a daily menu UI comprising subsets of the second set of food items in the second set of food item displays, configured by inputs to a temporal UI selector and a nutritional information display displaying a first set of nutritional values of the subsets of the second set of food items. The system displays a shopping list configuration UI comprising an item modification activator, a list modification activator, a list control activator, a shopping list display, and a second set of food item display comprising the second set of food items and quantities of the second set of food items. The system displays a food log configuration UI comprising a food log subset of the second set of food item displays configured by a temporal UI selector with a subset alteration UI activator and each food item comprising a food item quantity, a food item selector, a food item modification control UI activator, and a food item removal control. The system displays a recipe configuration UI comprising a generated recipe list display comprising a recipe descriptor, a recipe modification activator, a recipe removal control, and a recipe creator UI activator. The method switches between the daily menu UI, the shopping list configuration UI, the food log configuration UI, and the recipe configuration UI in response to inputs received through a UI view type selector/indicator.

In some configurations, the one or more menu influencer parameters comprise personal user health information and user health goals. In some instances, the food item influencers comprise an influencer type, a food item or food item category, and spatiotemporal influence selectors for a date/day and/or meal.

In some configurations, each subset of the second set of food item displays comprises a food item alteration UI activator, a food item removal control, and an item swap UI activator for each food item. The system may also receive an input for the item swap UI activator of a particular food item. The system may display an item swap UI for the particular food item comprising a food item nutrient comparison display with nutritional values of the particular food item in comparison to a candidate replacement food item generated by inputs received to a food item type filter, a food item quantity selector, a food item selector display, and a food item alteration control activator. The system may replace the particular food item from the subset of the second set of food items with the candidate replacement food item in response to receiving an input to the food item alteration control activator.

In some configurations, the daily menu UI comprises a nutrition intake display displaying a second set of nutritional values configured by inputs from a menu view selector and at least one nutrient subcategory display, each nutrient subcategory display comprises a list nutrient items with nutrient item selectors. The daily menu UI may display an enhanced nutrient item display comprising a third set of nutritional values configured by at least one nutrient item selector and the menu view selector.

In some configurations displaying the shopping list configuration UI involves displaying a shopping list configurator UI in response to an input to the list control activator. The system may also display a temporal subset of the second set of food item displays with corresponding temporally adjusted food item quantities in response to receiving temporal parameters through the shopping list configurator UI.

In some configurations, the food log configuration UI comprises a food log nutritional information display including a set of nutritional values configured by modifications to the food items in the food log subset of the second set of food item displays.

In some configurations, the system displays a recipe creator UI comprising at least one of recipe description inputs, a recipe alteration activator, food item configurators, a visibility control selector, recipe instruction input and combinations thereof.

In some configurations, a third party (healthcare provider/nutrition coach) portal may be provided to allow the third party individual to view summaries of individual food logs and activities periodically. From this access point, a professional may also modify rules used in menu presets and allow suggestions for changing levels of activity and diet. Furthermore, the system may be configured to add references, to use automated chat bots or similar communications capabilities, to offer in process encouragement, or to enable easy communication by the provider/coach with the user. In connection with this, the system may also provide capabilities that may be extended to take into account an individual's electronic medical record available through capabilities of at least one existing electronic medical record (EMR) systems.

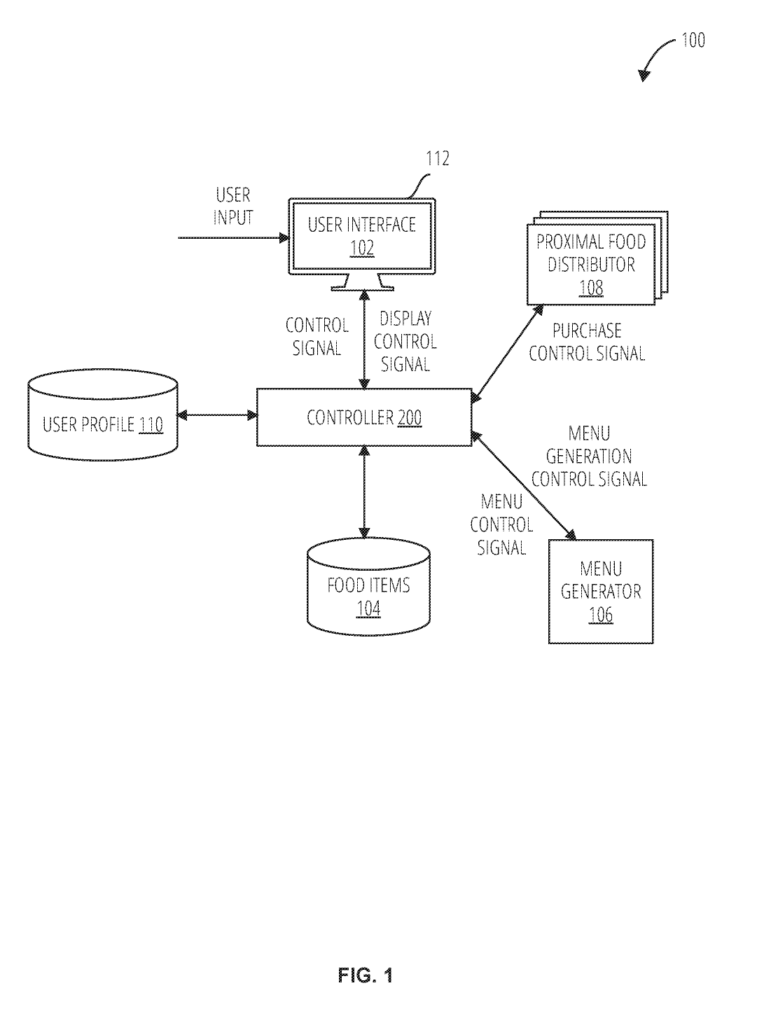

Referring to FIG. 1, the system 100 comprises a user interface 102, a proximal food distributor database 104, a menu generator 106, proximal food distributor 108, a user profile control memory data structure 110, a machine display 112, and a controller 200.

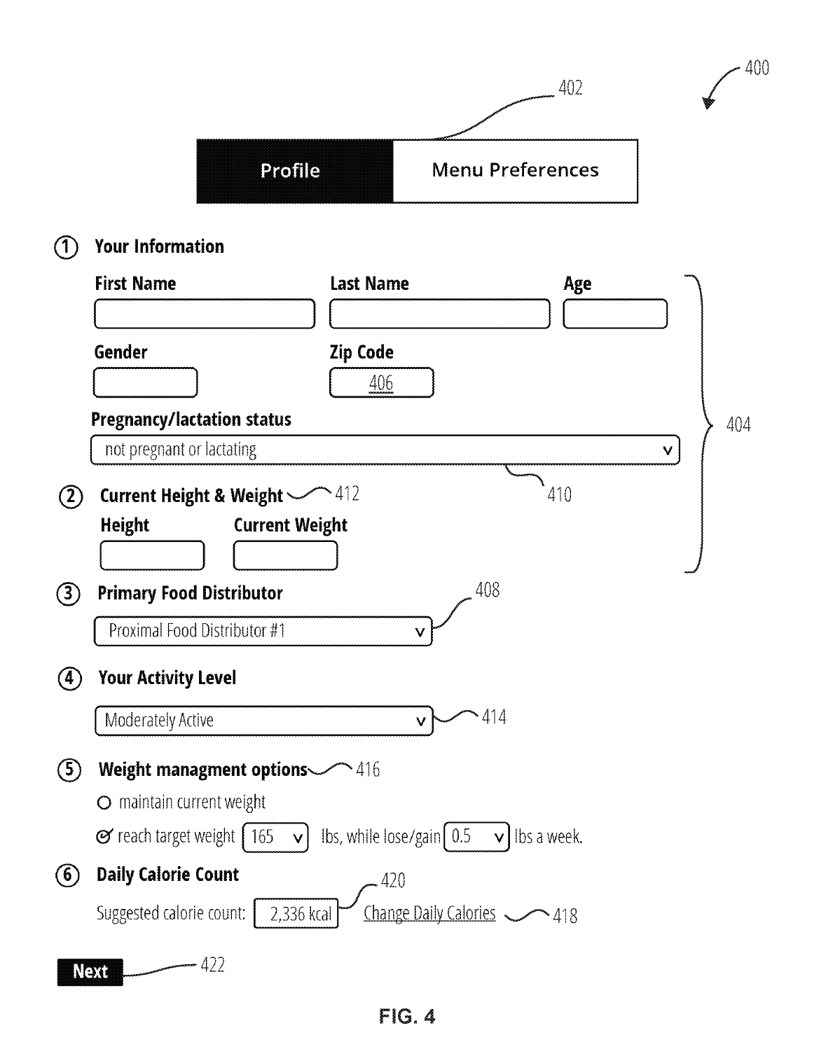

The user interface 102 is displayed on the machine display 112. The user interface 102 may receive a user input, which includes audio input, text input, haptic input, etc. The input may be at a control interface, which operates to send a control signal to the controller 200. The control signal may differ based on the control interface activated. The user interface 102 may be altered by the machine display 112 when a display control signal is received from the controller 200. Various user interfaces are depicted in FIG. 4 through FIG. 15.

The proximal food distributor database 104 stores one or more food items. The proximal food distributor database 104 may be altered by an input. The proximal food distributor database 104 receives a control signal from the controller 200 to send the one or more food items to the controller 200. The control signal may request all of the one or more food items or a subset of those one or more food items. The proximal food distributor database 104 sends the requested one or more food items to the controller 200. The one or more food items in proximal food distributor database 104 may be altered to include categories, tags, etc. to be utilized during retrieval of the one or more food items or a first set of food items. The categories/tags may include stores that sell the food item, brands, food type, cost, cuisine, etc. The proximal food distributor database 104 may comprise one or more control memory structures, which may include the one or more of the proximal food distributor 108. In some embodiments, the one or more food items that may comprise the one or more proximal food distributor 108 may be utilized as the proximal food distributor database 104.

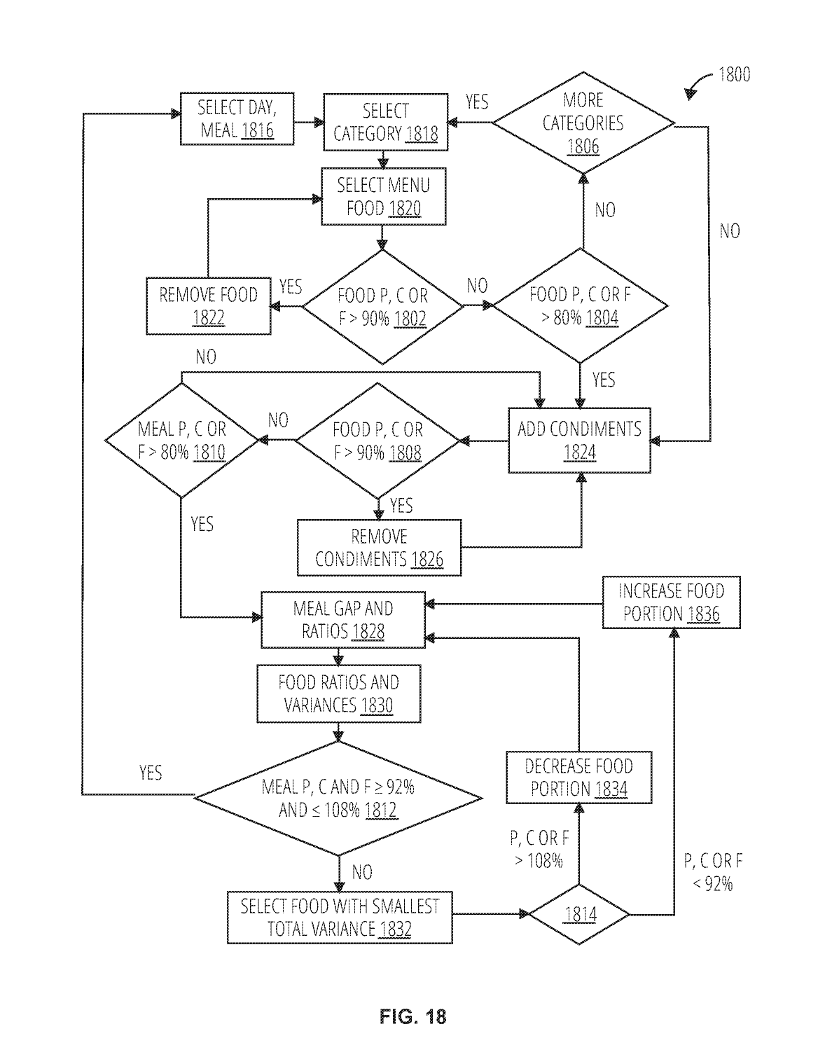

The menu generator 106 receives a menu generation control signal from the controller 200. The menu generator 106 utilizes the menu generation control signal, which comprises a first set of food items and a menu rules control, as an input to generate a food menu. The menu generation control signal may provide the number of food items to select, restrictions for each food item to be selected, and the available food item to be selected. An exemplary embodiment of a menu generator 106 is depicted in FIG. 18, which illustrates a process in the prior art.

The proximal food distributor 108 may receive a purchase control signal from the controller 200. The purchase control signal may be generated in response to the menu control signal. The purchase control signal may also be generated in response to the activation of a control of the user interface 102, the control being generated in response to the menu control signal. In some embodiments, the proximal food distributor 108 may operate as the proximal food distributor database 104, determining the one or more food items. One or more of the proximal food distributor 108 may be selected by the controller 200 to act as one or more of the one or more filters. The proximal food distributor 108 may be selected in response to an input to the user interface 102.

The user profile control memory data structure 110 may store information associated with each user. The information stored in the user profile control memory data structure 110 may be utilized to alter the operation of the controller 200. The user profile control memory data structure 110 may help determine the one or more food items received from the proximal food distributor database 104 and the proximal food distributor 108 utilized. The user profile control memory data structure 110 may also associate multiple users, altering the menu generation control signal.

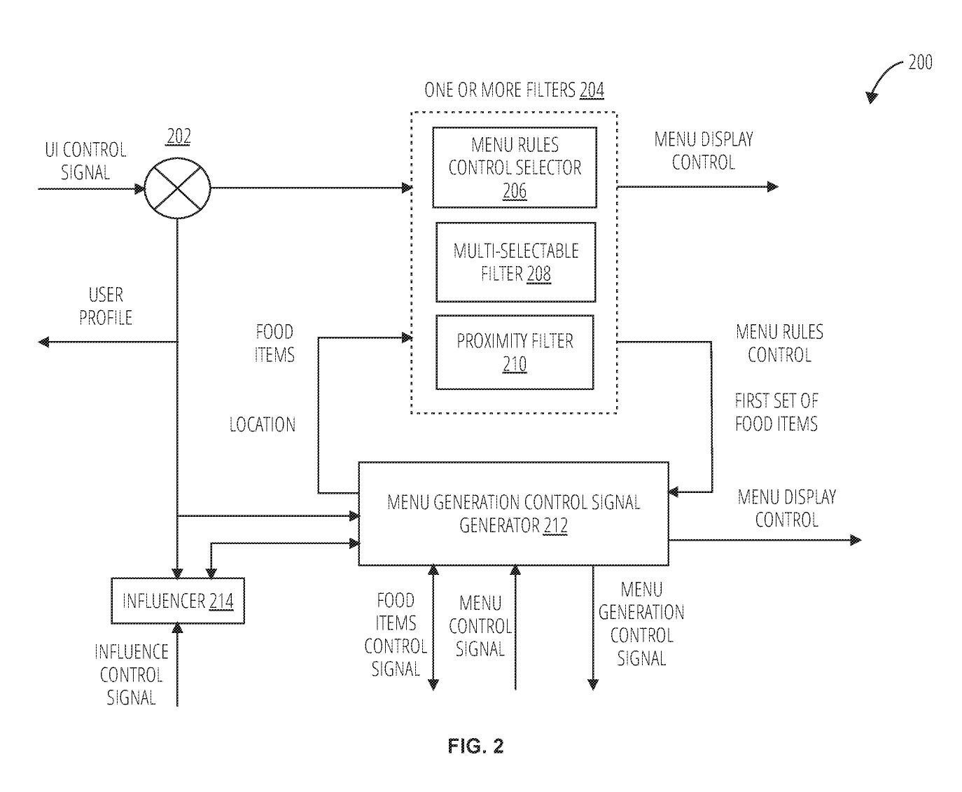

The machine display 112 may be part of a machine capable of displaying the user interface 102 and receiving inputs associated with the user interface 102, such as a personal computer, portable personal computer, etc. The controller 200 is described in reference to FIG. 2.

Referring to FIG. 2, the controller 200 comprises a selector 202, a one or more filters 204, a menu generation control signal generator 212, and an influencer 214. The one or more filters 204 may comprise a menu rules control selector 206, a multi-selectable filter 208, and a proximity filter 210.

The selector 202 receives a control signal from the user interface 102. The selector 202 determines the component of the controller 200, or external component, to which to send the control signal. The components may include the one or more filters 204, the menu generation control signal generator 212, the influencer 214, and the user profile control memory data structure 110.

The one or more filters 204 receive the control signal from the selector 202. The machine state of the one or more filters 204 are then altered based on the control signal. The one or more filters 204 may then send a menu display control signal to the machine display 112 to alter the user interface 102. The one or more filters 204 also receive information such as food items and location from the menu generation control signal generator 212. Based on the information and the machine state of the one or more filters 204, the one or more filters 204 sends the menu rules control and the first set of food items to the menu generation control signal generator 212. In another embodiment, the one or more filters 204 sends the machine state to the menu generation control signal generator 212, which may utilize the machine state to generate a food items request control signal to retrieve the first set of food items from the proximal food distributor database 104.

The menu rules control selector 206 receives a control signal to alter the machine state to select a menu rules control. In some embodiments, altering the machine state of the menu rules control selector 206 results in a display control signal that alters the user interface 102 to not display controls that alter the menu rules control selector 206. In addition, in some embodiments, the activation of one machine state of the menu rules control selector 206 precludes activation of another machine state of the menu rules control selector 206. The menu rules control selector 206 may also be altered by information received from the user profile control memory data structure 110 (e.g., via the menu generation control signal generator 212). The menu rules control is sent to the menu generation control signal generator 212. The menu rules control may provide the number of food items to select, restrictions for each food item to be selected, and the available food item to be selected. In some embodiments, the menu rules control determines a number of days for which to generate meals (e.g., breakfast, lunch, dinner, snacks, etc.), along with the numbers of food items to include in each meal. The menu rules controls may include such categories as “diabetic friendly”, “heart healthy”, “low cost”, etc., each may be utilized to alter the machine state of the menu rules control selector 206 in a different manner. The menu rules controls may be altered, and additional menu rules controls may be generated to reflect various nutritional interests of users or entities, such as grocery stores, healthcare providers/insurers, corporate cafeterias, food manufacture, non-profits, etc.

The multi-selectable filter 208 receives a control signal. The control signal alters the machine state of the multi-selectable filter 208 such that the machine state affects the transformation of the one or more food items into the first set of food items. The multi-selectable filter 208 may be altered by multiple control signals, resulting in a different machine state. A display control signal may be generated to alter the user interface 102 to display controls to select or de-select the filters associated with the multi-selectable filter 208.

The proximity filter 210 may receive a location from a control signal or the user profile control memory data structure 110. The control signal may include various inputs to the controller 200, including location markers tied to specific store locations, such as a WiFi identifier, a Quick Response code identifier, or other geomarker, which may be received by a device (including mobile devices) and sent to the controller 200. The proximity filter 210 determines the food items within a pre-determined or dynamically determined distance from the location. The proximity filter 210 may determine the grocery stores or food item warehouses within the pre-determined or dynamically determined distance from the location. The proximity filter 210 then affects the transformation of the one or more food items into the first set of food items. The proximity filter 210 may receive a control signal associated with a second location, and in some embodiments send a control signal to the menu generation control signal generator 212 to send a second menu generation control signal to the menu generator 106 to generate a second menu control signal utilizing the second location.

The menu generation control signal generator 212 sends a food item request control signal to the proximal food distributor database 104 and receives a food items control signal in response. In one embodiment, the food items control signal may comprise the one or more food items. In one embodiment, the food items control signal comprises the first set of food items (e.g., the food item request control signal comprises the machine state of the one or more filters 204). The menu generation control signal generator 212 may send the one or more food items and location to the one or more filters 204 and receive a menu rules control and first set of food items in response. The menu generation control signal generator 212 may utilize the menu rules control and first set of food items to generate a menu generation control signal. The menu generation control signal generator 212 may further receive a control signal from the influencer 214 to alter the menu generation control signal to alter the likelihood of one or more of the first set of food items to be selected by the menu generator 106 to be included in the second set of food items. The menu generation control signal generator 212 sends the menu generation control signal to the menu generator 106 and receives a menu control signal in response. The menu control signal comprises the second set of food items. The menu generation control signal generator 212 may send the menu generation control signal in response to receiving a control signal from the user interface 102. The menu generation control signal generator 212 sends a display control signal to the machine display 112 to alter the user interface 102 to display the second set of food items. The menu generation control signal generator 212 may receive further control signals to alter the second set of food items.

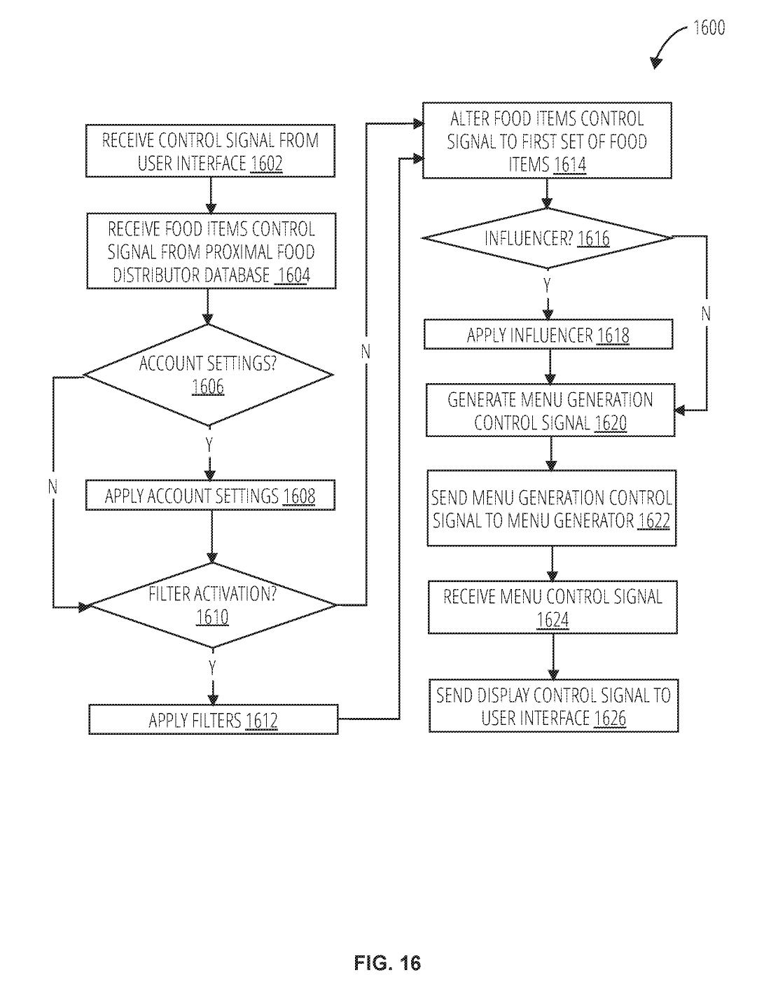

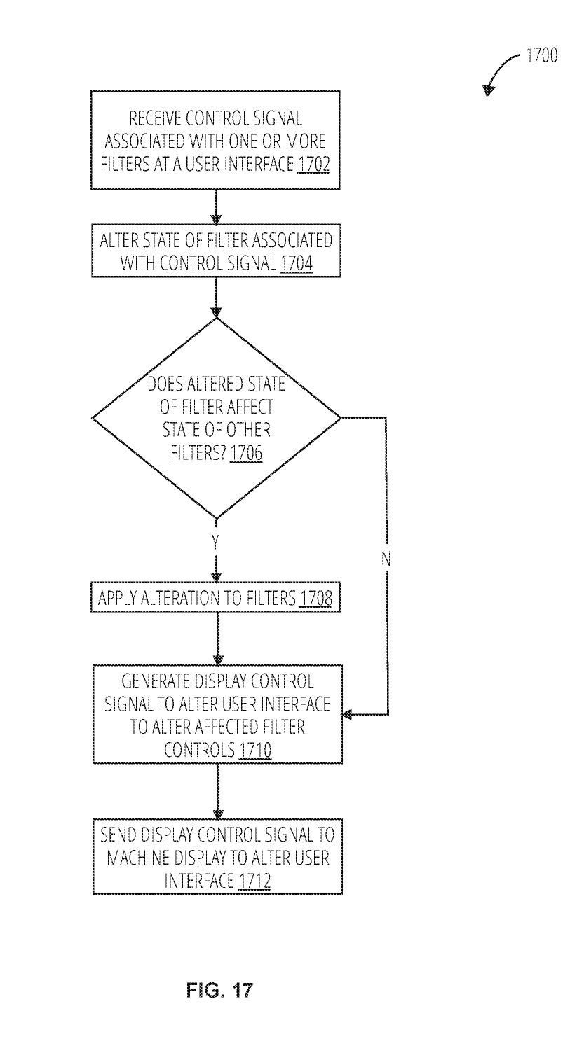

The influencer 214 receives control signals from the user interface 102, the user profile control memory data structure 110, and from external components. The control signals are sent to the menu generation control signal generator 212 to alter the likelihood of a one or more of the first set of food items being selected by the menu generator 106. The controller 200 may be operated in accordance with FIG. 16 and FIG. 17.

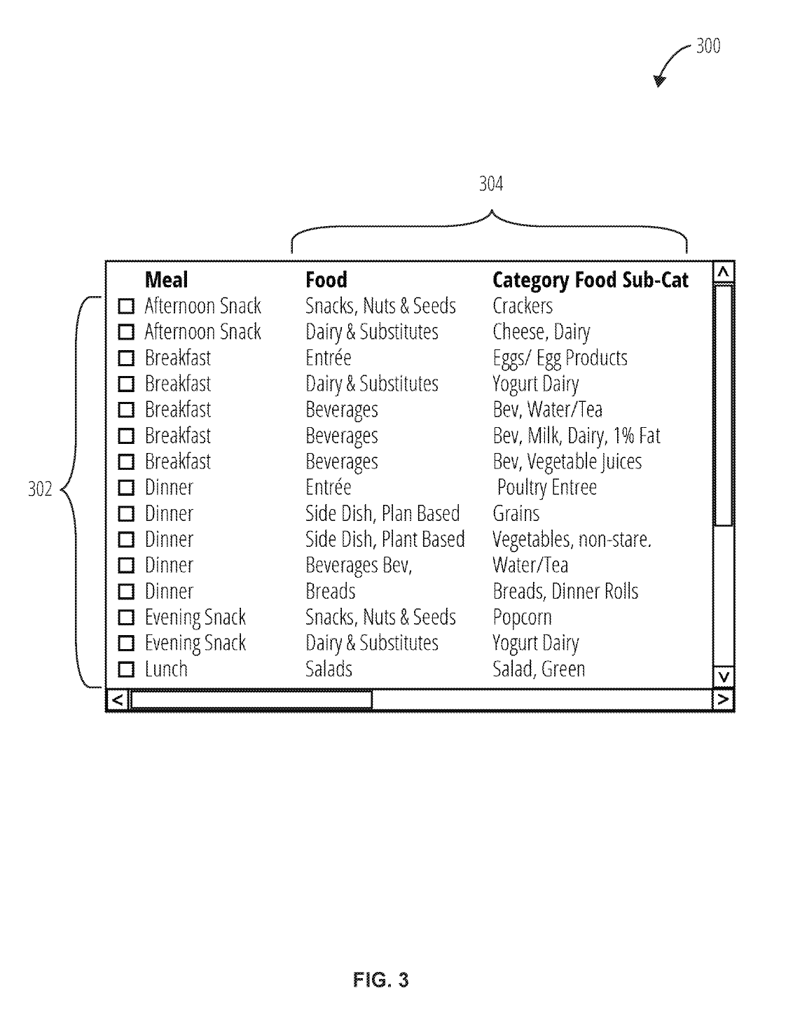

Referring to FIG. 3, the menu rules control 300 comprises a food item slots 302 and a food item restrictions 304. The food item slots 302 determine the number of food items to be selected for the second set of food items. The food item restrictions 304 determine the categories of food items from the first set of food items to be selected. For each of the food item slots 302, one food item is selected based on the first set of food items, which may be based on the proximal food distributor database 104 utilized and the machine state of the one or more filters 204. The menu generator 106 determines the second set of food items based on the inputs to the user interface, including the nutritional goals, the one or more filters selected, influences, etc. As depicted, the menu rules control 300 comprises food item slots 302 for one day. In other embodiments, the menu rules control 300 may include multiple days, which may have food items selected either dependently or independently. The menu rules control 300 may utilize various categories of time periods for which to generate a second set of food items.

Referring to FIG. 4, a user profile configuration UI 400 comprises a profile/preference indicator 402, a user profile information inputs 404, a location input 406, a menu influencer parameters 410, a menu influencer parameter 412, a proximal food distributor database selector 408, a menu influencer parameter 414, a menu influencer parameters 416, a daily target kcal 420, a menu influencer parameter 418, and a control signal activator 422.

The profile/preference indicator 402 is a user interface selector that indicates that the user profile configuration UI 400 is currently displaying user profile configuration options. The user profile configuration options include user profile information, primary food distributor, and menu influencing parameters. The user profile information inputs 404 receive user information such as name, age, gender, and location information through a location input 406 utilized in the generation of the user profile. The user profile information inputs 404 may also include personal user health information corresponding to specific menu influencing parameters such as the height and weight of the user (menu influencer parameter 412). In some configurations, the user profile information may include gender specific personal user health information inputs relevant to nutritional requirements not associated specifically with age, such as pregnancy and lactation which may also be configured as menu influencing parameters (menu influencer parameters 410).

The menu influencer parameter 414 provides a selector for receiving indication of a user's physical activity level. A user's physical activity level is personal user health information that changes the user's caloric requirements and serves as an menu influencing parameter. The menu influencer parameters 416 are user health goals (here, weight loss goals) that include a target weight and a target weight loss rate. The target weight and weight loss rate influence selection and quantity of food items and portion sizes for generation of the menu.

The location input 406 receives an input associated with the user's location such as a zip code. The input to the location input 406 may alter the available inputs to the proximal food distributor database selector 408. The user profile information inputs 404 receive inputs associated with identifying information of a user for generating the user profile user. The proximal food distributor database selector 408 is altered by the input to the location input 406. The location information is utilized to determine a subset of the proximal food distributor database 104 or to select one or more proximal food distributor database 104 to be utilized. In some embodiments, the proximal food distributor database selector 408 may be utilized to select proximal food distributors, as well as specific warehouses for food distributors based on the zip code. The grocers and warehouses may be utilized to determine the proximal food distributor database 104. The user profile configuration options may additionally include information generated from the inputs received from the user profile information inputs 404, the menu influencer parameter 414, and the menu influencer parameters 416 in the form of the daily target kcal 420. A configurable menu influencer parameter 418 is positioned adjacent to the daily target kcal 420 and allows modification of the display target kcal influencing selection and quantity of the food items in the menu generation.

In some configurations, the menu influencer parameter 412, the menu influencer parameter 414, the menu influencer parameters 416, the menu influencer parameter 418 may serve as a parameter set where combinations of particular values and/or value ranges in each parameter correspond to certain health related categories that may be utilized to activate nutrient guidelines for menu generation. For example, a health partner may have very detailed diets for a certain category of patients, with different micronutrient targets dependent upon a multitude of health measures.

The control signal activator 422 receives an input and in response sends a control signal to the controller 200. The control signal may also be sent to the user profile control memory data structure 110 in some embodiments. When the control signal activator 422 receives an input the user interface displays switches to menu preferences configuration UI 500 showing menu preference options.

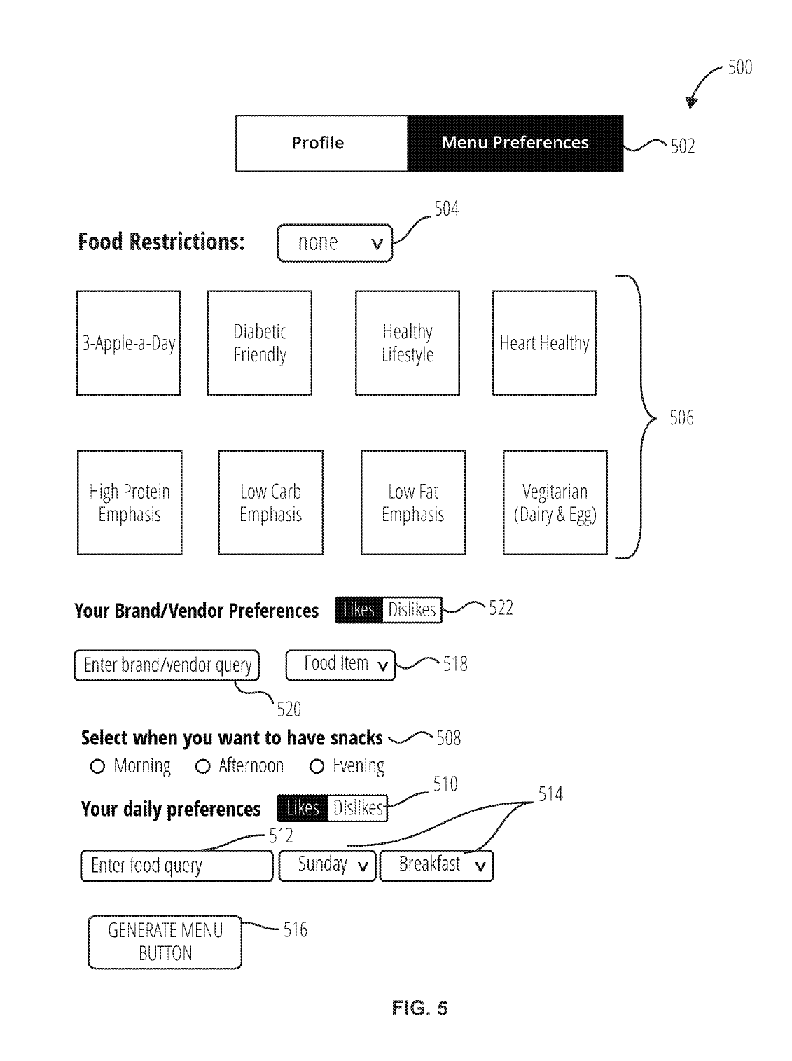

Referencing FIG. 5, a menu preferences configuration UI 500 comprises the profile/preference indicator 502, one or more menu rules control activators 506, a menu filter interface selector 504, temporal food item preference selector 508, an influencer type selector 510, a spatiotemporal influence selectors 514, a control signal activator 516, and food item influencer selectors 512. The menu preferences configuration UI 500 displays menu preferences options as indicated by profile/preference indicator 502. A menu filter interface selector 504

The menu preferences configuration UI 500 comprises a menu filter interface selector 504 (i.e., filter selector) that may receive an input to generate a control signal for a menu filter to alter the multi-selectable filter 208 to a base machine state, the base machine state being no filters selected. The filter selectors may receive an input to generate a control signal to alter the multi-selectable filter 208 to a machine state in which the associated filter is selected. The menu filter interface selector 504 may provide filter options for dietary restrictions that may include filter options for gluten free, dairy free, and vegan for example.

The one or more menu rules control activators 506 may receive an input to select the menu rules control that relates to a specific dietary theme. The menu rules control activators 506 correspond to menu rule controls that configure for specific diet styles in generated menus. In some configurations, these dietary themes may include a three apple a day diet, a diabetic friendly diet, a healthy lifestyle diet, a heart healthy diet, a high protein emphasis diet, a low carb emphasis diet, a low fat emphasis diet, and a vegetarian (dairy & egg) emphasis diet.

In response to the selection of a menu rules control, the menu preferences configuration UI 500 may be altered to preclude selection of a second of the menu rules control activators 506. The menu preferences configuration UI 500 may be altered to a state whereby selection of another menu rules control activators 506 results in the de-selection of the previously selected of the menu rules control activators 506.

The menu filter interface selector 504 may receive an input to alter the menu preferences configuration UI 500 to receive further inputs associated with the one or more filters. The one or more filters may correspond to food restriction settings that may include restrictions such as gluten-free, dairy free, and/or vegan. The influencer type selector 510 configures like or dislike (influencer type) of a particular food item identified by the food item influencer selectors 512. The spatiotemporal influence selectors 514 receive inputs associated with food likes and dislikes for specific days and meals times in order to influence meals generated in menu.

The temporal food item preference selector 508 provides the option for designating when the user would like to include a snack in their daily menu. The options include morning, afternoon, and evening times for when the user would like a snack.

The menu preferences configuration UI 500 comprises influencer type selector 510, food item influencer selectors 512, and spatiotemporal influence selectors 514. The food item influencer selectors 512 may be associated with one or more food items in a proximal food distributor database. The food item influencer selectors 512 may receive an input to associate the influence type activated by the influencer type selector 510 with one or more food items. The influence type selector may receive an input to alter the user interface to associate an increased likelihood that a food item associated with the selected influence selectors is selected by the menu generator or a decreased likelihood that a food item associated with the selected influence selectors is selected by the menu generator. The influence activator receives an input to send a control to the influencer 214 or the user profile control memory data structure 110 to associate the food item and the selected influence. The menu preferences configuration UI 500 may receive an input to alter the menu preferences configuration UI 500 to receive further inputs associated with the generation of further food items from current food items.

The control signal activator 516 may receive an input to generate a control signal to activate the controller 200 to send a menu generation control signal to the menu generator. The selected one of the menu rules control activators 506, the one or more filters selected utilizing the user interface associated with the menu filter interface selector 504, the influence control signals selected utilizing the user interface associated with the influencer type selector 510, and the further food items generated utilizing the user interface associated with the influencer type selector 510, the food item influencer selectors 512, and the spatiotemporal influence selectors 514 are utilized to generate the menu generation control signal.

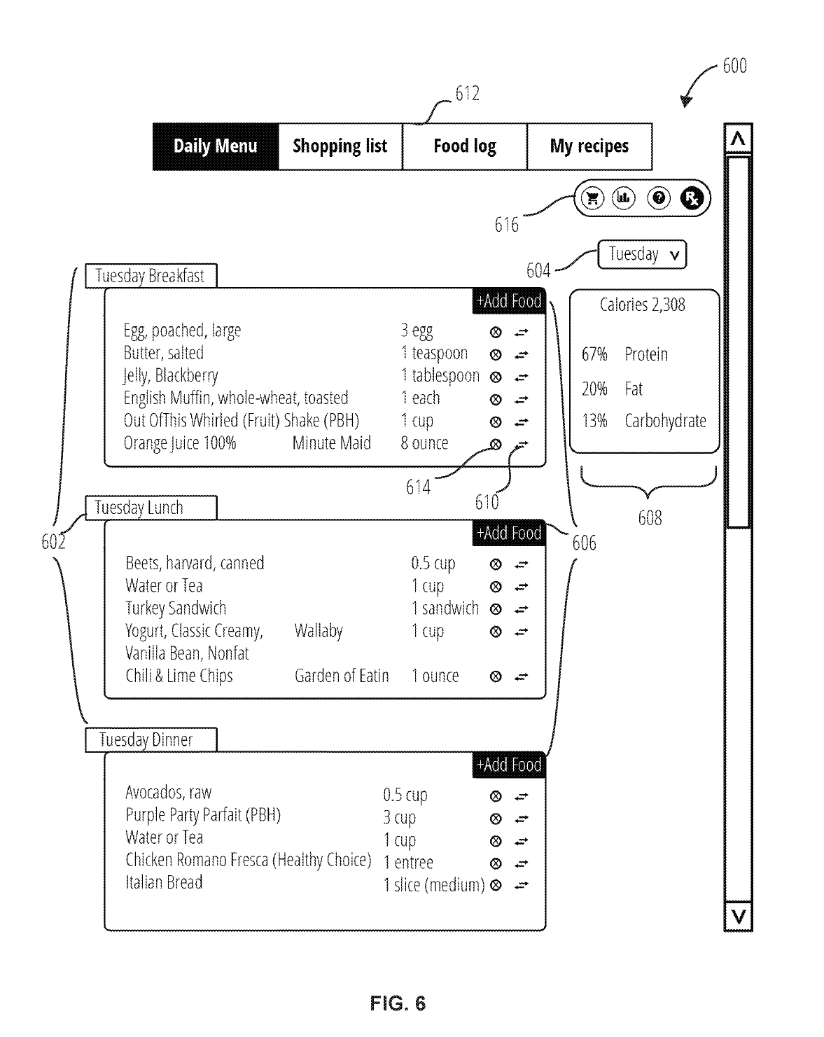

Referring to FIG. 6, a daily menu UI 600 displays a menu generated by the menu generator utilizing inputs from the user profile information inputs 404, the proximal food distributor database selector 412, the menu influencer parameter 414, the menu influencer parameters 416, the menu filter interface selector 504, the menu rules control activators 506, the influencer type selector 510, the food item influencer selectors 512, and the spatiotemporal influence selectors 514.

The daily menu UI 600 comprises a UI view type selector/indicator 612, a UI activators 616, a temporal UI selector 604, a nutritional information display 608, a subset of the second set of food items display 602 comprising a second set of food items alteration UI activators 606, a food item removal control 614, and an item swap UI activator 610. The UI view type selector/indicator 612 allows a user to navigate through a daily menu UI 600, a shopping list configuration UI 1000, a food log configuration UI 1300, and a recipe creator UI 1400 corresponding to a daily menu interface, a shopping list item interface, a food log interface, and a list of user specified recipes (“My Recipes”) interface, respectively. In the daily menu UI 600, the UI view type selector/indicator 612 indicates that the daily menu interface is being displayed.

The subset of the second set of food items display 602 displays a daily food menu configured by the input received through the temporal UI selector 604. The temporal UI selector 604 may receive an input for a specific date or day of the week in order to display the corresponding subset of the second set of food items display 602. The subset of the second set of food items display 602 comprises a display individual food items, portions sizes, and in some cases the food producer. Each food item includes a food item removal control 614 and an item swap UI activator 610. When the food item removal control 614 receives an input, the associated food item is removed from the subset of the second set of food items display 602. When the item swap UI activator 610 receives an input, item swap UI 900 is displayed as an overlay above daily menu UI 600 showing nutritional comparison information between the associated food item and another food item that may replace it.

Each second set of food item displays corresponds to a configured meal during the day/date shown by the temporal UI selector 604. In the daily menu UI 600, the subset of the second set of food items display 602 shows the food items for Tuesday's breakfast, lunch, and dinner with each meal time corresponding to a second set of food items displays. Each second set of food item displays includes a second set of food items alteration UI activators 606 that may allow the user to add an additional food item to the second set of food item displays.

In some configurations, alterations to the food items in the subset of the second set of food items display 602 (i.e., removal, swapping, and adding food items) may trigger the menu generator to run again utilizing the current food items, the user preferences, food influencers, and the menu preferences associated with the user.

A nutritional information display 608 is displayed adjacent to the subset of the second set of food items display 602 display information indicating the nutritional information associated with the daily menu displayed to the user. In some configurations, the nutritional information display 608 displays a first set of nutritional values corresponding to macronutrient information such as the percentage of protein, fat, and carbohydrates and the kilocalories associated in with the subset of the second set of food items display 602.

The daily menu UI 600 also includes a UI activators 616 comprising four selectable icons that activate additional options. Starting from the left, in one embodiment, clicking on the 1^{st} icon of the shopping cart will display the Shopping List screen with all its features identified in paragraphs 0088 through 0091. Clicking on the 2^{nd} icon of the vertical bar graph will display the Nutrient Analysis screen with all its features identified in paragraphs 0084 through 0085. Clicking on the 3^{rd} icon of the “?” will display a video identifying how to navigate this specific screen and the features of the specific screen the user is currently viewing. Clicking on the 4^{th} icon showing “Rx” will display a popup window with text reminding the user to be aware of food-drug interactions, and prompting the user who is on any medication to see a grocer pharmacist regarding any possible food-drug interactions from taking their medication(s). In other embodiments, clicking on the “Rx” icon will display details about potential negative food, medication, and supplement interactions based on the meal plan, clinical record, and user profile.

In some configurations, the subset of the second set of food items display 602 depicts part of a second set of food items displayed on a machine display and organized into a categorical list format. The temporal UI selector 604 is utilized to alter the daily menu UI 600 to display one or more other parts of the second set of food items. As depicted, the temporal UI selector 604 may display each day generated by the menu generator. The second set of food items alteration UI activators 606 receives an input to alter the daily menu UI 600 to display a control to alter one or more food items in the second set of food items. The nutritional information display 608 displays the nutritional information for the selected subset of the second set of food items display 602.

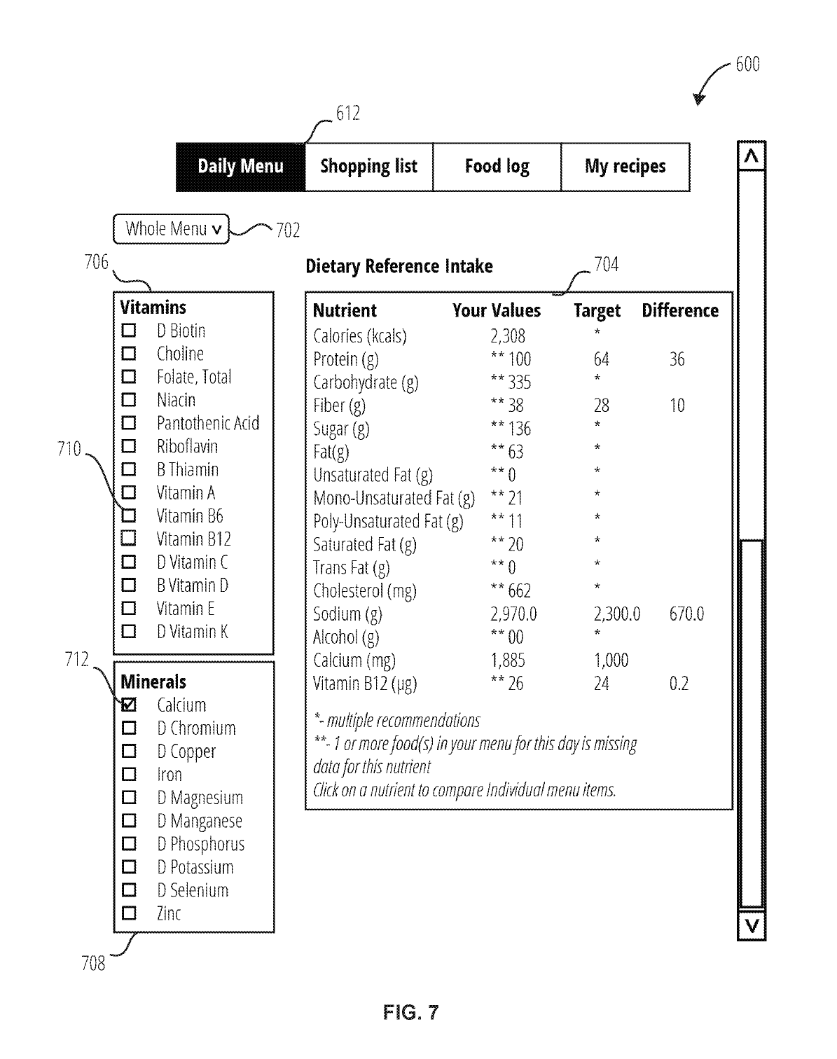

FIG. 7 shows a continuation of the daily menu UI 600 displaying a menu view selector 702, a nutrition intake display 704, a nutrient subcategory display 706, and a nutrition subcategory display 708. The menu view selector 702 operates as a selectable filter for displaying nutritional information from the generated menu. The menu view selector 702 filters may filter nutritional information for the entire menu, for a single day of the menu, or for a specific meal of the menu. In some configurations, the nutritional information is displayed through a nutrition intake display 704 as a second set of nutritional values showing the breakdown of the current nutrient intake for the menu against some recommended dietary guidelines. The nutrient subcategory display 706 shows nutrient item selector 710 for each nutrient allowing the user to inspect how much each food item in a subset of the second set of food items has for the particular nutrient. In the daily menu UI 600, the nutrient subcategory display 706 displays vitamins, each with a nutrient item selector 710. The nutrition subcategory display 708 displays minerals, each with a nutrient item selector 712. In some configurations, receiving an input for a nutrient item selector 712 generates the display of an enhanced nutrient item display 802 as an overlay above the nutrition intake display 704.

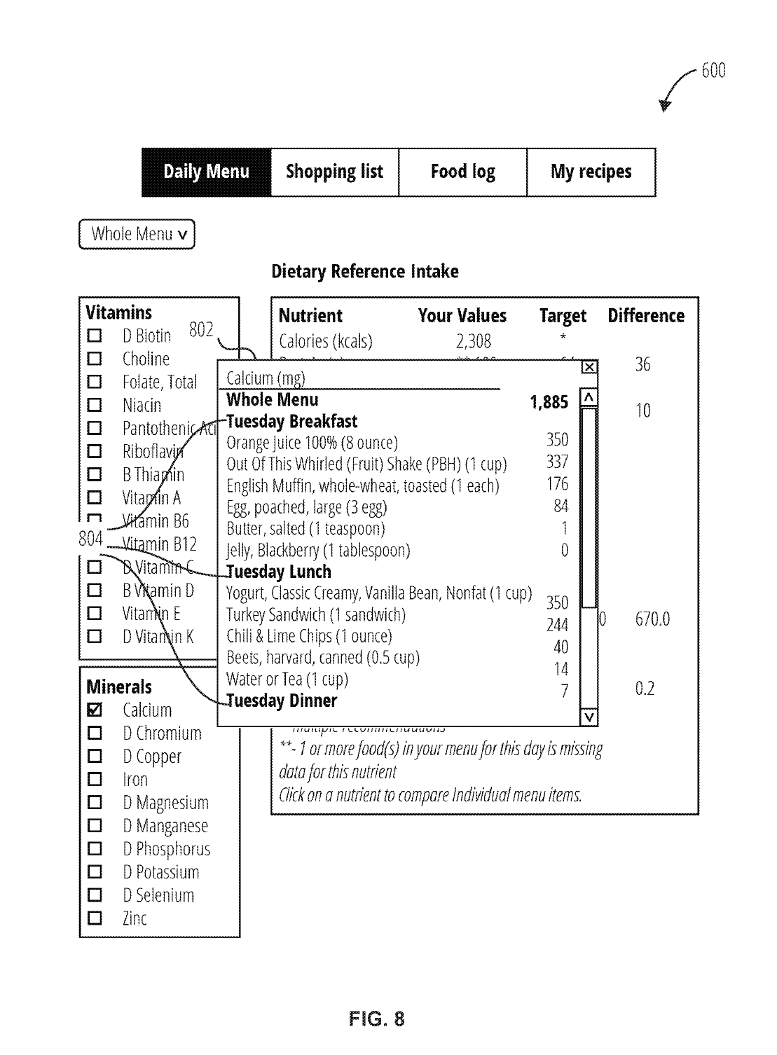

FIG. 8 displays an enhanced nutrient item display 802 shown as an overlay for a selected nutrient item type selector. In some configurations, the selection of any nutrient displays the contribution of each menu item to that nutrient for that day. In FIG. 8, the selected nutrient item type was calcium. The enhanced nutrient item display 802 displays a third set of nutritional values showing the breakdown of calcium across the whole menu in milligrams and the breakdown of calcium according to each item in the daily menu for that day in the nutrient menu item subcategories 804. In some configurations, multiple nutrient items may be selected and displayed in the same manner for the user.

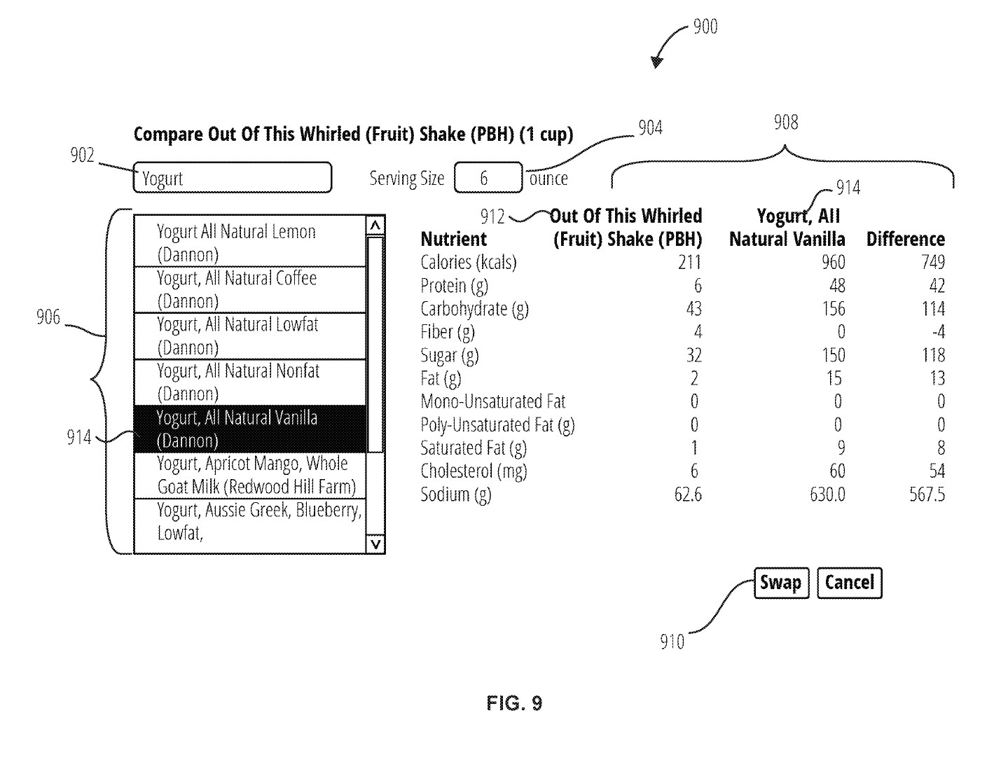

FIG. 9 displays an item swap UI 900 in response to an input being received for the item swap UI activator 610. The item swap UI 900 is displayed as an overlay above the daily menu UI 600, showing nutritional comparison information between the associated food item and another food item that may replace it. The item swap UI 900 comprises a food item selector display 906, a food item nutrient comparison display 908, and a food item alteration control activator 910.

The food item type filter 902 receives inputs that filter food items from the proximal food distributor databases. In the item swap UI 900, the input filters for “yogurts” and returns food items to display in the food item selector display 906. The food item selector display 906 display information associated with the selected food item such as the name of the food item, the producer, and some properties. A food item quantity selector 904 portions out the quantity of the particular food item to compare the particular food item 912 to a candidate replacement food item 914 in the food item selector display 906. The food item nutrient comparison display 908 compares the nutrient categories for the user. The food item nutrient comparison display 908 receives an input to activate a control to alter the item swap UI 900 to select additional food item(s) to compare to the displayed food item in the food item selector display 906. The food item alteration control activator 910 receives an input to activate a control to alter the second set of food items replacing the particular food item 912 with the candidate replacement food item 914. In some embodiments, any food item may be utilized to replace a food item in the second set of food items. In other embodiments, the menu control signal comprises alternate food items to the selected food items in the second set of food items that may be selected. In further embodiments, activation of the food item alteration control activator 910 generates a control for the controller 200 to send a menu generation control signal to the menu generator 106.

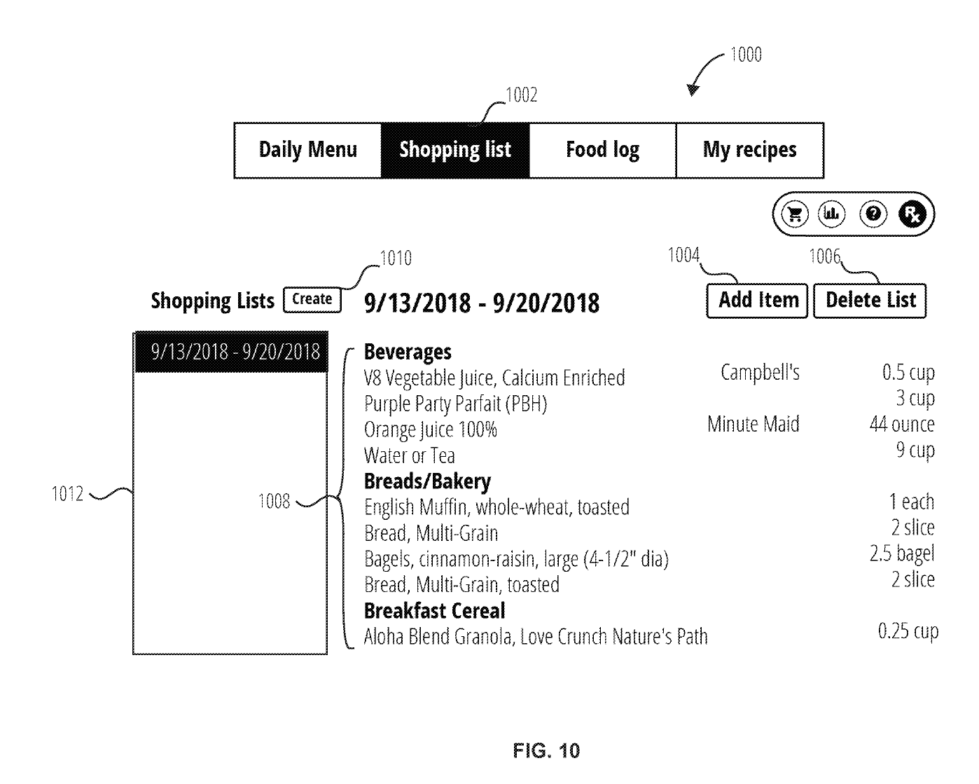

FIG. 10 shows a shopping list configuration UI 1000 comprising a UI view type selector/indicator 1002, an item modification activator 1004, a list modification activator 1006, a second set of food item display 1008, a list control activator 1010, and a shopping list display 1012. The UI view type selector/indicator 1002 indicates that the shopping list user interface is being displayed. The shopping list shows a list of food items in the second set of food item display 1008 at least comprising food item categories, the food item name, and food item quantity. The shopping list display 1012 lists available shopping lists. The current shopping list displayed in shopping list configuration UI 1000 is for the date range of 9/13-9/20 and is the only highlighted list. Adding items to the shopping list may be initiated in response to receiving an input to the item modification activator 1004. Removal of the shopping list may be initiated in response to receiving an input through the list modification activator 1006.

The shopping list configuration UI 1000 shows the shopping list user interface that reflects seven-days' worth of menu items. A user may edit the amounts or name of the item, delete any item, and add any food or non-food item to the list.

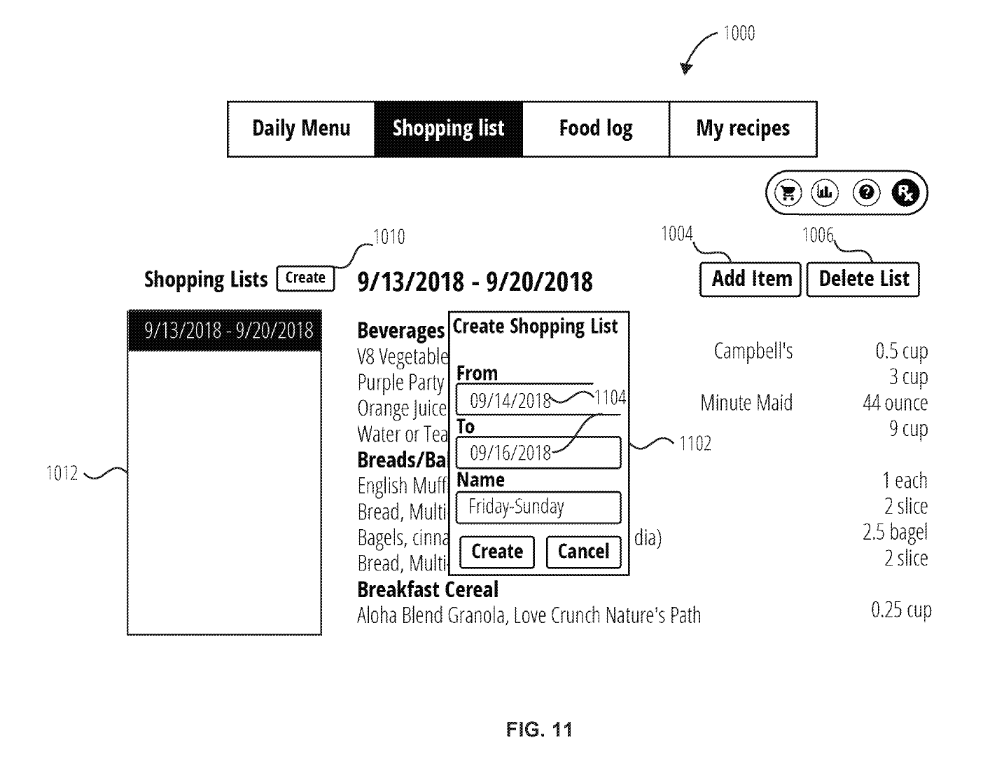

FIG. 11 shows the shopping list configuration UI 1000 with a shopping list configurator UI 1102 displayed as an overlay. In some instances, a user may wish to divide shopping lists with smaller date ranges. This may be done by some users to ensure that certain food items are consumed within a few days of purchase. In order to add another shopping list, an input would be received through the list control activator 1010, and a shopping list configurator UI 1102 comprising temporal parameters 1104 is displayed as an overlay to create a new shopping list for a particular set of days. In some configurations, the user can select date ranges (temporal parameters 1104) for the shopping list that recreate the shopping list for a smaller subset of days. For example, if the current shopping list displayed is for an entire week between 9/13-9/20, the user may enter a new date range for the date range specifying that they want the current items in the list to be for the date range 9/14-9/16, and with the name of the shopping list included as “Friday-Sunday”. The creation of the shopping list adjusts the food quantity of the food items that a user wishes to purchase.

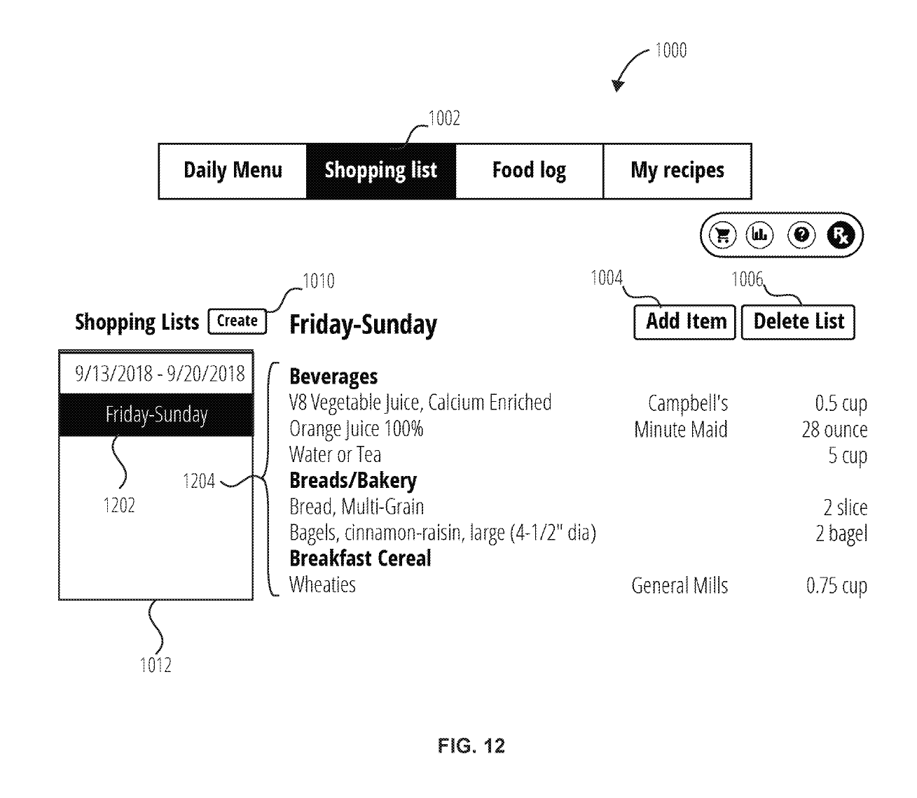

FIG. 12 shows the shopping list configuration UI 1000 after the shopping divided shopping list is created for the date range 9/14-9/16 with the name “Friday-Sunday”, the shopping list configuration UI 1000 shows a selected list display view 1202 for the newly created shopping list and displays the adjusted food items and quantities (temporally adjusted food item quantities) in a temporal subset of the second set of food item display 1204.

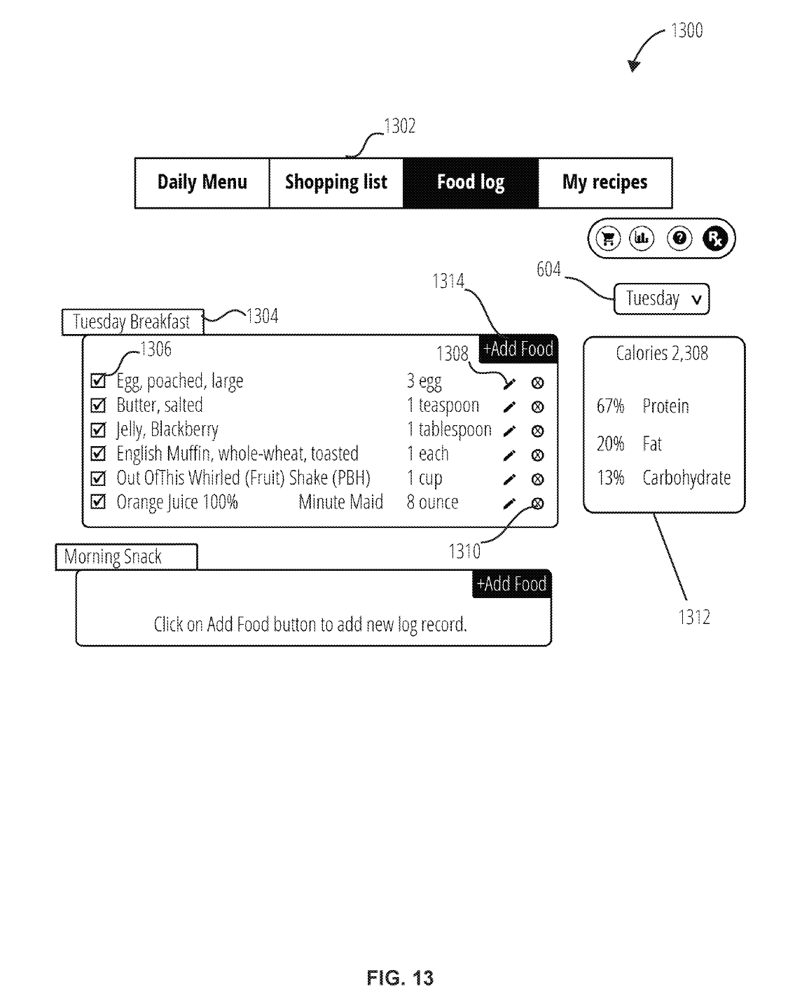

FIG. 13 shows a food log user interface to the user. The UI view type selector/indicator 1302 indicates that the food log user interface has been selected by the user and displays a temporal UI selector 604, a nutritional information display 1312, and at least one food log subset of the second set of food items display 1304 corresponding to a particular meal during the day selected by the temporal UI selector 604. Each of the food log subset of the second set of food items display 1304 displays food items populated from the generated menu, and is initially the same food items as seen in the daily menu user interface. Each food item includes a food item selector 1306 that indicates if the user consumed the particular food item for the particular meal. The user may self-report whether they consumed the food item, by leaving the food item with a check mark in the food item selector 1306. The user may also remove the food item by unselecting the check box in the food item selector 1306 or by selecting the food item removal control 1310. The user may also edit the quantity consumed of the food item by selecting the food item modification control UI activator 1308 and entering the correct quantity. The subset alteration UI activator 1314 may be selected by a user to add an additional food item and quantity to the subset of the second set of food items display 1304. The nutritional information display 1312 displays the nutrient information (nutrient and caloric information) corresponding to the food items the user reports they consumed. Initially, the nutritional information display 1312 displays the expected calories consumed based on the user's generated menu. When food items are removed, added, and/or edited, the displayed quantities of nutrients and calories are adjust to reflect the changes. The food item selector may, in other embodiments, be activated by a voice control, food item photo input, etc.

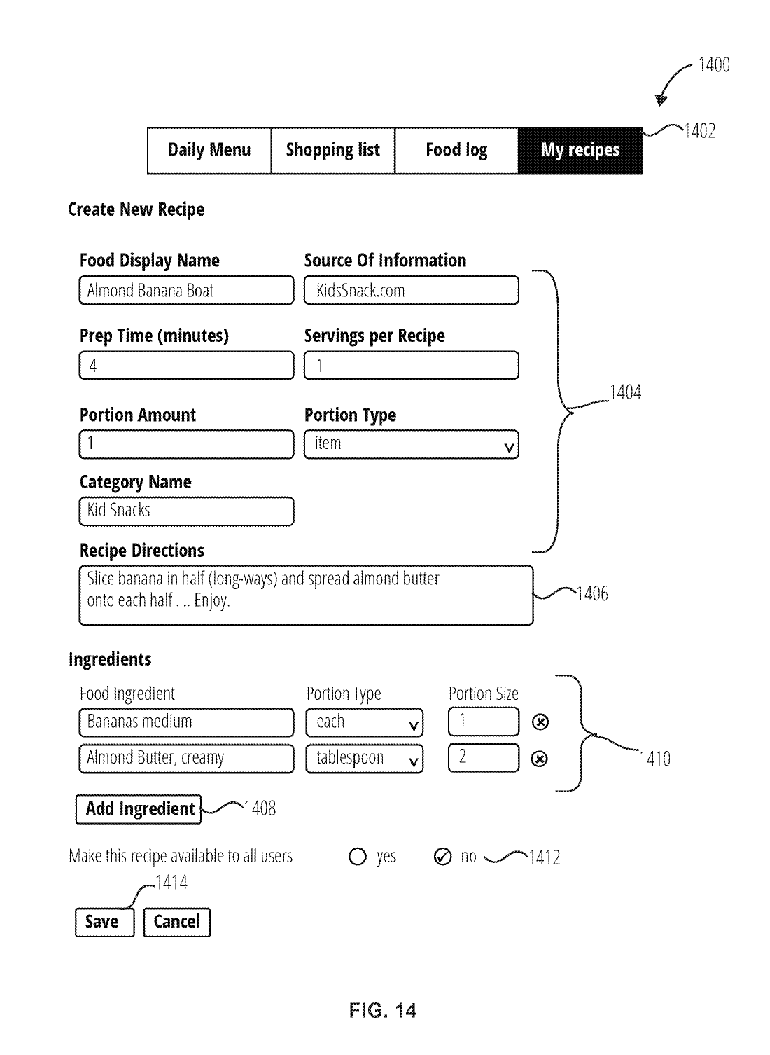

Referring to FIG. 14, the recipe creator UI 1400 displays the “my recipes” user interface. In some configurations, the recipe creator UI 1400 may provide users with a “create new recipe” user interface, depending on the presence of existing recipes. The “create new recipe” user interface comprises recipe description inputs 1404, a recipe instruction input 1406, a recipe alteration activator 1408 that generates food item configurators 1410, a visibility control selector 1412, and a recipe control activator 1414. The recipe description inputs 1404 comprise inputs for entering the name of the recipe, identification of the recipe source, the preparation time for the recipe, the servings for the recipe, the portion amount for each serving, the portion type of the serving, and a category name for the recipe. The recipe instruction input 1406 receives inputs corresponding to instructions for creating the recipe. The food item configurators 1410 list the constituent food items that make up the recipe as well as the portion type for each food item and the portion size. The recipe alteration activator 1408 receives inputs to generate each of the food item configurators 1410 during the creation of a new recipe. A visibility control selector 1412 is provided to the user to allow their recipes to be searched by other users. The user can select to make the recipe visible to others or decline the access to other users. When all items of the “create new recipe” user interface have been completed, the recipe control activator 1414 may receive an input and generate the recipe.

In some configurations, nutritional information includes components such as micro-nutrients, macro-nutrients, glycemic index or load, flavones, phenols, and other components that are categorized as bio-active compounds, Phyto-nutrients, and Phyto-chemicals. The food selector is capable of taking into account any food component known now or in the future to have an impact on individual health.

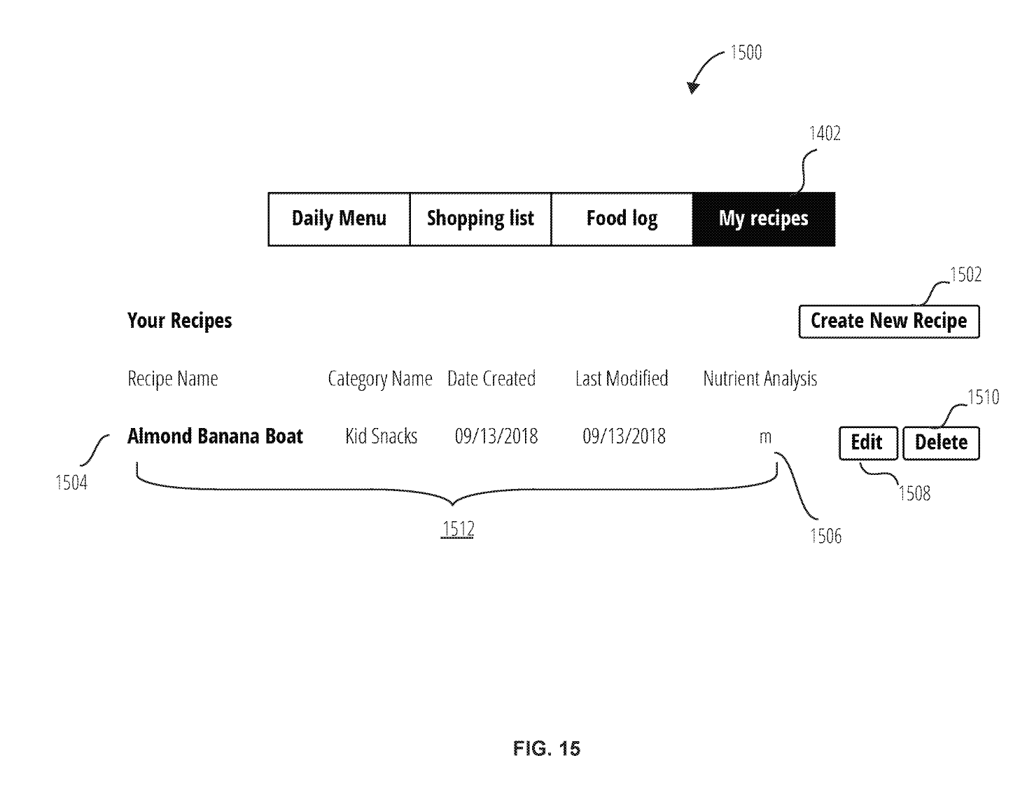

FIG. 15 displays recipe creator UI 1400 displaying a list of recipes created by the user. The recipe creator UI activator 1502 receives an input to generate an additional recipe. The existing generated recipes may be stored in the user profile control memory data structure 110. The recipe description inputs 1404 may populate information of the recipe descriptor 1512 in a generated recipe list display 1504. The displayed recipe in the generated recipe list display 1504 may be edited in a UI activated upon receiving an input through the recipe modification activator 1508. The recipe may be deleted upon receiving an input through the recipe removal control 1510. The recipe food item nutritional display activator 1506 receives an input to alter the recipe creator UI 1400 to display the nutritional information of the generated recipe utilizing the food items entered in the food item configurators 1410 for the associated recipe.

Referring to FIG. 16, the meal plan generation process 1600 receives a control signal from a user interface (block 1602). A food items control signal is received from a proximal food distributor database (block 1604). In some embodiments, the food items control signal is received in response to a food items request control signal being sent to a proximal food distributor database. The meal plan generation process 1600 determines whether there are associated account settings (decision block 1606). The account setting may be stored in an account control memory structure. If so, the account settings are applied (block 1608).

If not or after applying the account settings, the meal plan generation process 1600 determines whether one or more filters are activated (decision block 1610). The one or more filters may be activated by the process depicted in FIG. 17. If so, the one or more filters are applied (block 1612). The food items control signal is transformed into a first set of food items (block 1614). The menu rules control may also be determined.

The meal plan generation process 1600 then determines whether there is an influencer (decision block 1616). If so, the influencer is applied. The influencer may alter the likelihood of a food item in the first set of food items to be selected to be included in the second set of food items.

If not or once applied, a menu generation control signal is generated (block 1620). The menu generation control signal may be generated from the first set of food items, the account settings, the menu rules control, and the applied influences. The menu generation control signal is then sent to the menu generator (block 1622). The menu generation control signal may comprise instructions to generate a menu control signal, the menu control signal comprising a second set of food items, and send the menu control signal to the meal plan generation process 1600. The menu control signal is received (block 1624). The menu control signal may comprise the second set of food items. A display control signal is then sent to the user interface (block 1626). The display control signal comprises instructions to display the second set of food items.

In another embodiment, the one or more filters are utilized to generate a control to send to the proximal food distributor database, the first set of food items retrieved from the proximal food distributor database in response.

Referring to FIG. 17, the filter alteration method 1700 receives a control signal associated with one or more filters at a user interface (block 1702). The machine state of the one of the one or more filters associated with the control signal is altered (block 1704). The filter alteration method 1700 then determines whether the altered state of the altered filter affects the state of other filters (decision block 1706). If so, the alteration is applied to those filters (block 1708). For example, activating a first filter may preclude a second filter from being activated. A display control signal is generated comprising instructions to alter the user interface to alter the affected filter controls (block 1710). The display control signal is sent to the machine display to alter the user interface (block 1712).

Referring to FIG. 18, in one embodiment the menu generator process 1800 may comprise selecting a day, e.g., Monday, Tuesday, etc., and a meal for the day such as a breakfast, lunch, dinner or snack to be filled (block 1816). A category may also be identified for the meal, such as an entree, side dish, beverage or desert (block 1818). After identifying a category, a component to the category may be selected at random from a pre-established list therefor (block 1820). Examples of components to categories include, for example, an egg for an entree, a carbonated drink for a beverage, or yogurt as a side dish. Pre-established lists may be available for establishing the various categories and components. They may further be associated for relationship to publicly available databases, proprietary databases, or specialty databases of, for example, given vendors or food-manufactures.

In some configurations, the system may use of a proprietary database comprising food items and information from multiple public and private sources. For example, the USDA provides one source of food items on a public database. Other sources may include food manufacturers, either directly or via a third-party repository. A query to the food database may produce information of a particular food item such as, for example, a chicken breast with or without bones. The weight may be entered as three-ounces to establish a quantity of the food item. Conventional tools may provide further analysis of the food item to establish its nutritional breakdown. This type of analysis may be performed for a plurality of food items in a given meal.

In one embodiment, a menu may be divided into days and meals. The meals may be further divided into categories and components. This information may then be used, in one embodiment, to designate sub-groups or regions of the overall food database from which records may be pulled to fill the specified menu fields. In addition, elements pulled from the users' clinical record may be used to designate, or alternatively block out, regions of the food database during selection of food items when generating clinical menus for one or more individuals. In other words, the categories and components pre-established for a meal and in addition to attributes of the one or more individual's clinical record, may each individually, or alternatively and collectively, influence the availability of food items that may be available for selection from the food database. Other provisions of the clinical records, either alone or in combination with previous attributes, may then influence how to pick or chose food items from the previously defined pool or sub-region(s) during the generation of the clinical menu.

A menu generator and method of menu generation may automatically create clinical menus based on pre-established clinical criteria of the one or more individuals. A user, such as a dietitian, may previously establish a number of daily kilo-calories targeted for the one or more individuals and a number of grams of protein, fat and carbohydrate targeted for the one or more individuals within the kilo-calorie budget. The dietitian may further structure the number of days for the menu. The client may also indicate categories for the meal and meal patterns within the menu cycle. The criteria may further include food preferences, prescriptions and/or likes/dislikes or intolerances of the one or more individuals.

The menu generator, with these setup provisions, may construct a menu for the one or more individuals, one meal at a time day-by-day within the menu cycle. In generating the meals of the menu, the generator may select food items matching the selected categories and components while also balancing a distribution of grams of protein, fat, and carbohydrate that may have been previously tailored by a dietitian into guide-lines for meeting clinical needs of the one or more individuals.

In accordance with an example of an embodiment for a menu generator process 1800, a day and meal of the menu may be determined for selection of food items (block 1816). Next, categories may be identified for the meal, such as categories of an entree, beverage, side dish, dessert, etc. (block 1818). Additionally, components may be specified for the categories such as an egg component to an entree category from which a food item may then be selected, such as scrambled eggs (block 1820).

After selecting a food item, a query may determine if the food item initially selected may comprise more than 90% of the protein, carbohydrate or fat values designated from the clinical criteria (decision block 1802). If the food item would provide more than 90% of one of these macro-nutritional element guidelines, then the method removes the initially selected food item (e.g., scrambled eggs) (block 1822) and may select an alternative food, e.g., poached eggs (block 1820).

If the query determines a selected food item to meet the macro-nutritional guidelines, then the process may query to determine whether the meal thus constructed meets at least 80% of the protein, carbohydrate and fat objectives (decision block 1804). If the query determines that the meal contains less than 80% of these nutritional goals for the meal, then the generator may obtain more calories to meet the kilo-calorie budget for the meal (decision block 1806). The generation may then proceed to fill another category for the meal, e.g., to select a beverage for the meal (block 1818). If the meal should substantially meet the calorie budget established therefor, then the method may consider addition of a condiment (block 1824). Likewise, should the previous query determine that the food items of the meal thus constructed comprise nutritional elements exceeding 80% of the macro-nutritional guidelines, then the generation may similarly consider the addition of condiments (block 1824).

The generation of the clinical menu may perform “add condiments” (block 1824), query “food PCF>90%” (decision block 1808), and query “meal PCF greater than 80%” (decision block 1810) to add condiments to food item(s) of the meal until meal nutrients may meet at least 80% of the targeted nutritional guidelines. This routine may also aim to keep the food with condiments within the desired 90% values of the protein, carbohydrate and fat guidelines established therefor. If the food item with condiment additions should exceed a 90% threshold for proteins, carbohydrates or fats, then the generation may remove a condiment (block 1826) and select a replacement condiment (block 1824) or choose an alternative food for receiving added condiments.

In accordance with one embodiment, the food items that may be used for creating a menu may be obtained from available databases, such as the USDA Food Databases (e.g., Nutrient Database for Standard Reference and the USDA Branded Food Database). The USDA Food Databases may provide records to 247,326 items based on the April, 2018 major revision (and subsequent minor revisions). These menu foods may be further characterized with portions of minimum, typical, and maximum. The amount or portion of these menu foods may be increased or decreased by a given incremental amount until reaching the minimum or maximum levels. The nutrient content of food items may, thus, be derived from such exemplary food database.

It may be understood that alternative embodiments of the present invention may use food databases other than the USDA Nutrient Database, or may add additional food records or employ a combination of different databases, which may become available.

Additionally, in accordance with an embodiment of the present invention, each menu food of the food database may have one or more meal categories (i.e., entree, side dish, beverage, dessert, etc.) to which it may be associated or indexed. These categorizations, again as described previously herein, may assist in an allocation of regions or sub-regions of the database to be indexed and drawn upon for the selection of certain food provisions.

In a further embodiment, once a particular food type or component has been selected (i.e., egg, pork, etc.), it may then be blocked from selection to other categories of the meal and or menu. This may assist generation of a menu with varied food types in the make-up of a meal.

Furthermore, the categories may be prioritized to ensure that a meal fills more important elements first before filling others. These priorities may also be used to assist determination of particular food items that may be adjusted in portion size (discussed more fully herein below) or when designating a particular food item to remove from a meal.

Another embodiment may allow provision of categories and meals of prescribed (or Rx) food items. For example, a dietitian may prescribe the one or more individuals fish for dinners on Monday, Wednesday, and Friday. These prescribed foods may then be assigned into the prescribed categories and meals first, before synthesis of the rest of the menu, and may thus be viewed of utmost priority. The menu generation may take into consideration these prescribed food items when determining the nutritional guidelines for affecting the synthesis of the other meals and categories of the menu.

After filling the various categories of a meal with selected food items, menu synthesis may proceed to a refinement process. The refinement process may be described as performing an analysis and adjustment of Meal Gaps and Ratios.

Meal gaps and ratios may be determined after the various components for a meal have been populated with food items, and their nutritional breakdown determined to meet at least 80% of the macro-nutritional clinical criteria (block 1828). The “gap” may be defined as an absolute value of a nutrient target for a meal minus the actual value for the same nutrient element in the meal. For example, a protein gap may comprise the absolute value for proteins targeted for a meal minus the total proteins determined from an analysis of the meal just generated.

The “ratio” may be referenced alternatively as the “gap ratio.” In this embodiment, it may be defined as the gap for a given nutrient divided by the sum of all nutrient gaps. Additionally, this value may be multiplied by 100 and the ratio term may be described as a percentage. For example, a protein gap ratio may equal to:

GAP RATIOₚᵣₒₜₑᵢₙ=GAPₚᵣₒₜₑᵢₙ/(GAPₚᵣₒₜₑᵢₙ+GAP_{carbs}+GAP_{fat})×100 Equation 1

Gaps and Ratios may be determined for each macro-nutrient of the meal. In a further embodiment, these Gaps and Ratios may be stored in a pre-established table for the menu generation user or operator (such as the one or more individuals or dietitian). The Gaps and Ratios may be stored in the table associated with the respective meal and day.

After determining gaps and ratios, ratios and variances for the foods may be determined (block 1830). These may be determined by firstly, obtaining the macro-nutrient breakdown of each food. For each nutrient element, the food ratio may be calculated by taking the given nutrient determined for the food and dividing it by the sum of all its nutrients. For expression as a percentage, this result may be further multiplied by 100. For example, a food protein ratio may be calculated by the following:

Food Protein Ratio=Food Protein/(Food Protein+Food Carbs+Food Fat)×100 Equation 2

The variance for each nutrient for each food may then be determined by taking the absolute value of the food's Ratio minus the meal's Gap Ratio. Further to the above examples, the food protein variance may be represented by the expression:

Food Protein Variance=ABS(Food Protein Ratio−GAP RATIOₚᵣₒₜₑᵢₙ) Equation 3

These variances of the nutrients may be summed together to provide a total variance for the food. In particular embodiments, the food ratios, variances, and total variances may be stored in the table pre-established for the user and associated with the respective meals and day. These values may be recalled subsequently to assist in the analysis of and possible adjustments to menu meals.

After determining the variances, the current percentages of each macro-nutrient in the meal may be examined (decision block 1812). If all nutrients of the meal are within predetermined guidelines for the meal, then the meal may be described as meeting its target for each nutrient, and another meal may be generated (block 1816). It may be understood that if all meals of a day have been generated, the process will begin generating meals for a new day of the menu cycle. Likewise, it may be understood that if all meals for all days of the menu have been generated, then the menu generation may be complete.

In a particular embodiment, the nutritional guidelines may be set to a certain tolerance about a nutrient goal. For example, the tolerance in one embodiment may be set to about ±8%. For such an embodiment, proteins for a meal may be examined to determine if their levels are between 92 and 108% of the protein goal of the meal. Likewise, its carbohydrates and fats may be examined to determine if their levels fall within 92 and 108% of their respective targets. The meal may be described as meeting its macro-nutritional guidelines if the nutrients for the meal fall within ±8% of their respective goals. In alternative embodiments, these tolerances may be set to values other than ±8%. Additionally, the tolerances may comprise different levels for the various nutrients.

If a particular meal has not met its macro-nutritional guidelines, the menu generation may identify a food item in the meal that may be best suited for adjustment in order to bring nutrients of the meal within predetermined nutrient goals, e.g., ±8%. These food item(s) may then be adjusted in proportion, either increasing or decreasing their portions in the meal, so as to adjust the nutrient contents of the meal into conformity with its nutrient guidelines.

In one embodiment, a routine may be performed to analyze the previously determined food ratios to select a food item of nutritional make-up appropriate for making an impact upon the meal total if its portion is adjusted. For example, if the protein level of a meal may need adjusting, it may be more appropriate to adjust a portion of an egg item within the breakfast menu as opposed to making an adjustment to the portion of an orange juice beverage. In this example, an adjustment to the egg portion may provide a more effective impact on meal protein levels in comparison to adjustment of orange juice portions. Accordingly, the program may examine the food ratios to identify more than one food items that may be adjusted for affecting the nutrient levels. In a particular embodiment, three different food items may be identified for possible adjustment.

Next, continuing with a further embodiment, to determine which of the food items to select for adjustment of a given nutrient, the menu generation routine may examine the previously determined food variances (e.g., these values may be retrieved from the table pre-established for the user and associated with the food items and meal) and identify the food item of smallest variance for the particular nutrient (block 1832). If the percentage of the nutrient in the meal is determined (decision block 1814) to be greater than 108% of its target value, then the food item identified of the smallest variance for this particular nutrient may have its portion reduced (block 1834). Alternatively, if the level of this given nutrient in the meal is determined (decision block 1814) to be less than 92% of its target value, then the food item identified may have its portion increased (block 1836). After adjustment of portion of a food item in the meal, the meal analysis may return to calculation of Meal Gaps and Ratios (block 1828).

These procedures of determining Meal Gaps and Ratios (block 1828), food ratios and variances (block 1830), meal analysis for guideline compliance (decision block 1812) and possible adjustments (block 1832, decision block 1814, block 1834, and block 1836) may be repeated until obtaining a meal that may conform to the nutrient guideline. However, if a minimum or maximum portion of the food item has been reached or the food's portion cannot be adjusted without going below or above the nutrient's tolerance level, then a substitute food item may be selected for adjustment.

If, however, there is a need to select a food item of the meal, the program may determine if one of the foods exceeds the 108% nutritional guideline and may then substitute the food item with one that may provide a better fit to the meal's overall goals. The meal may again be analyzed by these procedures to determine meal gaps and rations, food ratios and variances, and analysis of meal nutritional conformity and possible portion adjustment.

In a further embodiment, an additional analysis and adjustment may be performed. The nutrient levels of the meal may again be examined and a percentage determined by which each nutrient might deviate from its target for the meal. If the percentage determined for a discrepancy of each nutrient is less than or equal to 8%, then the meal may be deemed complete, and an additional meal process may be pursued.

If a greater discrepancy, e.g., greater than 8%, is determined, then the program for menu generation may identify the nutrient of largest discrepancy and calculate a range in grams, for example, of the nutrient that may need to be added for bring the meal percentage within its 8% tolerance level. The most that the gram weights may adjusted may be defined by:

Adj_{min/max}=nutrient target±0.08(nutrient target)−nutrient total Equation 4

If a food item in the menu may be found to enable adjustment within the min/max adjustment range, which may provide an adjustment of the nutrient value within the desired guidelines, then the program may adjust the portion of the food identified for bring the meal within its nutrient guidelines. After such adjustment, the meal analysis may be repeated to verify compliance of the meal with the nutritional guidelines.

In a further embodiment, if a food item in the meal/snack may be found not suitable for adjustment with the min/max adjustment range, then the portions of all foods in the meal may be restored to their initial levels. The menu generations may pursue the calculation of the meal gaps and ratios, food ratios and variances, meal analysis, and possible food adjustment again but with preference to alternative foods for adjustment or replacement of select food items therefore with replacement that may be more in-line with the meal objectives.

Again, once a meal may be determined to be within its nutritional guidelines, the program may proceed to generate another meal of the menu. In accordance with one embodiment of the present invention, a method of menu generation may incorporate residual nutritional deviations of meals previously generated into the synthesis of follow-on meals. Such embodiments may adjust the nutritional targets for the subsequent meals in accordance with the previous residuals. By affecting the target values of the later meals dependent on the residuals resulting from the previously generated meals, an overall accumulated deviation per day (or menu cycle) may be kept to a minimum.

For example, a breakfast may first be generated with a protein level at 102% of its target value. When generating a lunch for the same day, the initial target value for proteins for the lunch may be reduced by 2%. Likewise, if the breakfast meal resulted with a −5% residual for carbohydrates, the program may adjust the carbohydrate target for the lunch by +5%. Accordingly, upon reaching the end of the day the combined nutritional breakdown of all meals for the day may be kept within ±8% of any one meal.

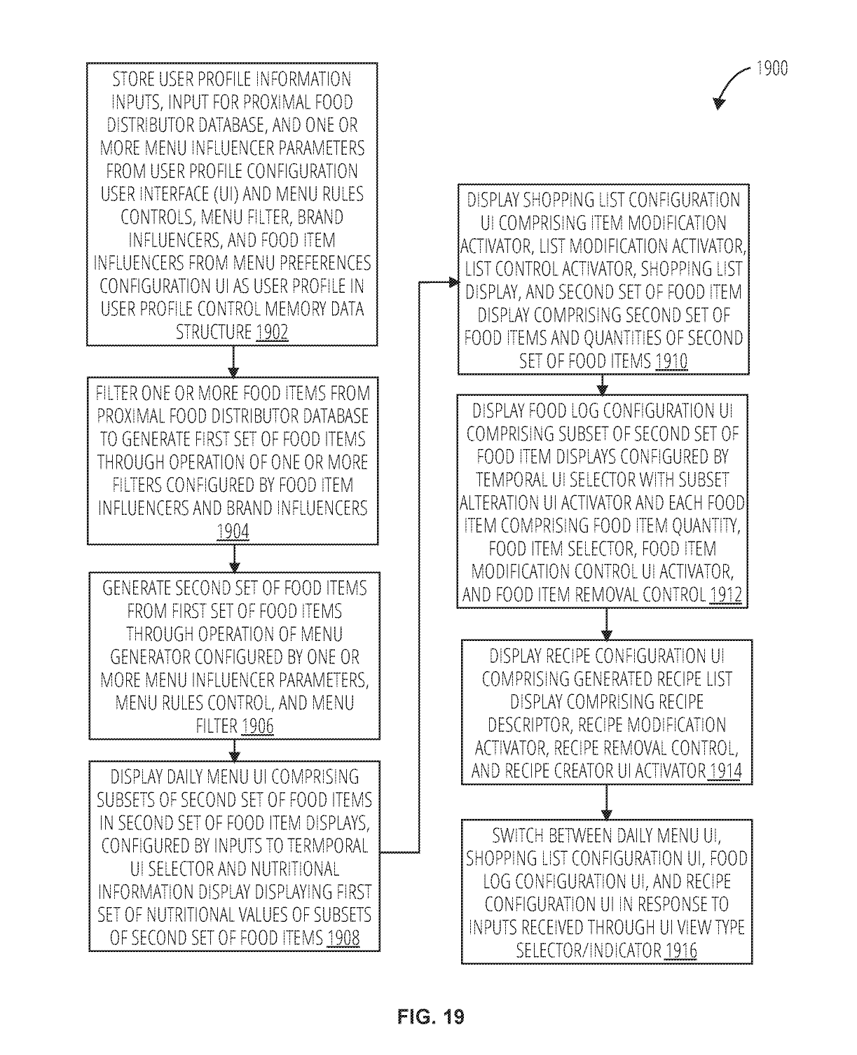

Referencing FIG. 19, in block 1902, method 1900 stores user profile information inputs, an input for a proximal food distributor database, one or more menu influencer parameters from a user profile configuration user interface (UI), menu rules controls, a menu filter, brand influencer, and food item influencers from a menu preferences configuration UI as a user profile in a user profile control memory data structure. The brand influencers and the food item influencers each comprise an influencer type. In block 1904, method 1900 filters one or more food items from the proximal food distributor database to generate a first set of food items through operation of one or more filters configured by the food item influencers and the brand influencers. In block 1906, method 1900 generates a second set of food items from the first set of food items through operation of a menu generator configured by the one or more menu influencer parameters, menu rules control, and the menu filter. In block 1908, method 1900 displays a daily menu UI comprising subsets of the second set of food items in a second set of food item displays, configured by inputs to a temporal UI selector and a nutritional information display displaying a first set of nutritional values of the subsets of the second set of food items. In block 1910, method 1900 displays a shopping list configuration UI comprising an item modification activator, a list modification activator, a list control activator, a shopping list display, and a second set of food item display comprising the second set of food items and quantities of the second set of food items. In block 1912, method 1900 displays a food log configuration UI comprising the subset of the second set of food item displays configured by a temporal UI selector with a subset alteration UI activator and each food item comprising a food item quantity, a food item selector, a food item modification control UI activator, and a food item removal control. In block 1914, method 1900 displays a recipe configuration UI comprising a generated recipe list display comprising a recipe descriptor, a recipe modification activator, a recipe removal control, and a recipe creator UI activator. In block 1916, method 1900 switches between the daily menu UI, the shopping list configuration UI, the food log configuration UI, and the recipe configuration UI in response to inputs received through a UI view type selector/indicator.

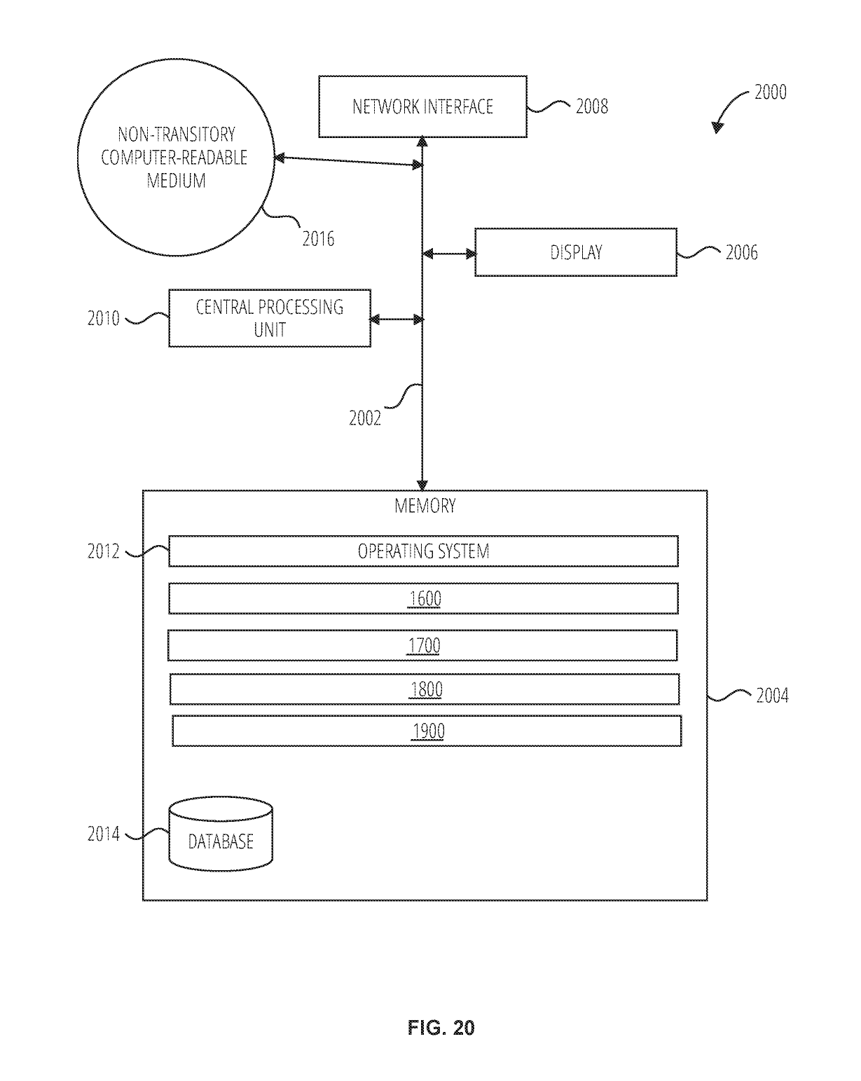

FIG. 20 illustrates several components of an exemplary system 2000 in accordance with one embodiment. In various embodiments, system 2000 may include a desktop PC, server, workstation, mobile phone, laptop, tablet, set-top box, appliance, or other computing device that is capable of performing operations such as those described herein. In some embodiments, system 2000 may include many more components than those shown in FIG. 20. However, it is not necessary that all of these generally conventional components be shown in order to disclose an illustrative embodiment. Collectively, the various tangible components or a subset of the tangible components may be referred to herein as “logic” configured or adapted in a particular way, for example as logic configured or adapted with particular software or firmware.

In various embodiments, system 2000 may comprise one or more physical and/or logical devices that collectively provide the functionalities described herein. In some embodiments, system 2000 may comprise one or more replicated and/or distributed physical or logical devices.

In some embodiments, system 2000 may comprise one or more computing resources provisioned from a “cloud computing” provider, for example, Amazon Elastic Compute Cloud (“Amazon EC2”), provided by Amazon.com, Inc. of Seattle, Wash.; Sun Cloud Compute Utility, provided by Sun Microsystems, Inc. of Santa Clara, Calif.; Windows Azure, provided by Microsoft Corporation of Redmond, Wash., and the like.

System 2000 includes a bus 2002 interconnecting several components including a network interface 2008, a display 2006, a central processing unit 2010, and a memory 2004.

Memory 2004 generally comprises a random access memory (“RAM”) and permanent non-transitory mass storage device, such as a hard disk drive or solid-state drive. Memory 2004 stores an operating system 2012.

These and other software components may be loaded into memory 2004 of system 2000 using a drive mechanism (not shown) associated with a non-transitory computer-readable medium 2016, such as a DVD/CD-ROM drive, memory card, network download, or the like.

Memory 2004 also includes database 2014. In some embodiments, system 2000 may communicate with database 2014 via network interface 2008, a storage area network (“SAN”), a high-speed serial bus, and/or via the other suitable communication technology.

In some embodiments, database 2014 may comprise one or more storage resources provisioned from a “cloud storage” provider, for example, Amazon Simple Storage Service (“Amazon S3”), provided by Amazon.com, Inc. of Seattle, Wash., Google Cloud Storage, provided by Google, Inc. of Mountain View, Calif., and the like.

Terms used herein should be accorded their ordinary meaning in the relevant arts, or the meaning indicated by their use in context, but if an express definition is provided, that meaning controls.

“Circuitry” in this context refers to electrical circuitry having at least one discrete electrical circuit, electrical circuitry having at least one integrated circuit, electrical circuitry having at least one application specific integrated circuit, circuitry forming a general purpose computing device configured by a computer program (e.g., a general purpose computer configured by a computer program which at least partially carries out processes or devices described herein, or a microprocessor configured by a computer program which at least partially carries out processes or devices described herein), circuitry forming a memory device (e.g., forms of random access memory), or circuitry forming a communications device (e.g., a modem, communications switch, or optical-electrical equipment).

“Firmware” in this context refers to software logic embodied as processor-executable instructions stored in read-only memories or media.

“Hardware” in this context refers to logic embodied as analog or digital circuitry.

“Logic” in this context refers to machine memory circuits, non-transitory machine readable media, and/or circuitry which by way of its material and/or material-energy configuration comprises control and/or procedural signals, and/or settings and values (such as resistance, impedance, capacitance, inductance, current/voltage ratings, etc.), that may be applied to influence the operation of a device. Magnetic media, electronic circuits, electrical and optical memory (both volatile and nonvolatile), and firmware are examples of logic. Logic specifically excludes pure signals or software per se (however does not exclude machine memories comprising software and thereby forming configurations of matter).

“Programmable device” in this context refers to an integrated circuit designed to be configured and/or reconfigured after manufacturing. The term “programmable processor” is another name for a programmable device herein. Programmable devices may include programmable processors, such as field programmable gate arrays (FPGAs), configurable hardware logic (CHL), and/or any other type programmable devices. Configuration of the programmable device is generally specified using a computer code or data such as a hardware description language (HDL), such as for example Verilog, VHDL, or the like. A programmable device may include an array of programmable logic blocks and a hierarchy of reconfigurable interconnects that allow the programmable logic blocks to be coupled to each other according to the descriptions in the HDL code. Each of the programmable logic blocks may be configured to perform complex combinational functions, or merely simple logic gates, such as AND, and XOR logic blocks. In most FPGAs, logic blocks also include memory elements, which may be simple latches, flip-flops, hereinafter also referred to as “flops,” or more complex blocks of memory. Depending on the length of the interconnections between different logic blocks, signals may arrive at input terminals of the logic blocks at different times.

“Software” in this context refers to logic implemented as processor-executable instructions in a machine memory (e.g. read/write volatile or nonvolatile memory or media).

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other. Any terms not expressly defined herein have their conventional meaning as commonly understood by those having skill in the relevant art(s).

Various logic functional operations described herein may be implemented in logic that is referred to using a noun or noun phrase reflecting said operation or function. For example, an association operation may be carried out by an “associator” or “correlator”. Likewise, switching may be carried out by a “switch”, selection by a “selector”, and so on.

Those skilled in the art will recognize that it is common within the art to describe devices or processes in the fashion set forth herein, and thereafter use standard engineering practices to integrate such described devices or processes into larger systems. At least a portion of the devices or processes described herein can be integrated into a network processing system via a reasonable amount of experimentation. Various embodiments are described herein and presented by way of example and not limitation.

Those having skill in the art will appreciate that there are various logic implementations by which processes and/or systems described herein can be effected (e.g., hardware, software, or firmware), and that the preferred vehicle will vary with the context in which the processes are deployed. If an implementer determines that speed and accuracy are paramount, the implementer may opt for a hardware or firmware implementation; alternatively, if flexibility is paramount, the implementer may opt for a solely software implementation; or, yet again alternatively, the implementer may opt for some combination of hardware, software, or firmware. Hence, there are numerous possible implementations by which the processes described herein may be effected, none of which is inherently superior to the other in that any vehicle to be utilized is a choice dependent upon the context in which the implementation will be deployed and the specific concerns (e.g., speed, flexibility, or predictability) of the implementer, any of which may vary. Those skilled in the art will recognize that optical aspects of implementations may involve optically-oriented hardware, software, and or firmware.

Those skilled in the art will appreciate that logic may be distributed throughout one or more devices, and/or may be comprised of combinations memory, media, processing circuits and controllers, other circuits, and so on. Therefore, in the interest of clarity and correctness logic may not always be distinctly illustrated in drawings of devices and systems, although it is inherently present therein. The techniques and procedures described herein may be implemented via logic distributed in one or more computing devices. The particular distribution and choice of logic will vary according to implementation.

The foregoing detailed description has set forth various embodiments of the devices or processes via the use of block diagrams, flowcharts, or examples. Insofar as such block diagrams, flowcharts, or examples contain one or more functions or operations, it will be understood as notorious by those within the art that each function or operation within such block diagrams, flowcharts, or examples can be implemented, individually or collectively, by a wide range of hardware, software, firmware, or virtually any combination thereof. Portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in standard integrated circuits, as one or more computer programs running on one or more processing devices (e.g., as one or more programs running on one or more computer systems), as one or more programs running on one or more processors (e.g., as one or more programs running on one or more microprocessors), as firmware, or as virtually any combination thereof, and that designing the circuitry or writing the code for the software or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies equally regardless of the particular type of signal bearing media used to actually carry out the distribution. Examples of a signal bearing media include, but are not limited to, the following: recordable type media such as floppy disks, hard disk drives, CD ROMs, digital tape, flash drives, SD cards, solid state fixed or removable storage, and computer memory.

What is claimed is:

1. A method comprising:
storing user profile information inputs, an input for a proximal food distributor database, and one or more menu influencer parameters from a user profile configuration, wherein a user interface (UI) and menu rules controls, a menu filter, brand influencers, and food item influencers from a menu preferences configuration UI as a user profile in a user profile control memory data structure, wherein the brand influencers and the food item influencers each comprise an influencer type;
filtering one or more food items from the proximal food distributor database to generate a first set of food items through operation of one or more filters configured by the food item influencers and the brand influencers;
generating a second set of food items from the first set of food items through operation of a menu generator configured by the one or more menu influencer parameters, the menu rules control, and the menu filter;
displaying a daily menu UI comprising subsets of the second set of food items in a second set of food item displays, wherein the daily menu UI is configured by inputs to a temporal UI selector and a nutritional information display displaying a first set of nutritional values of the subsets of the second set of food items;
displaying a shopping list configuration UI comprising an item modification activator, a list modification activator, a list control activator, a shopping list display, and a second set of food item display, wherein the second set of food item display comprises the second set of food items and quantities of the second set of food items;
displaying a food log configuration UI comprising a food log subset of the second set of food item displays comprise a subset alteration UI activator and food items a food item selector, a food item modification control UI activator, and a food item removal control, wherein the second set of food item displays being configured by a temporal UI selector and each food item comprising a food item quantity;
displaying a recipe configuration UI comprising a generated recipe list display, the generated recipe list display comprising a recipe descriptor, a recipe modification activator, a recipe removal control, and a recipe creator UI activator; and
switching between the daily menu UI, the shopping list configuration UI, the food log configuration UI, and the recipe configuration UI in response to inputs received through a UI view type selector.

2. The method of claim 1, wherein the one or more menu influencer parameters comprise personal user health information and user health goals.

3. The method of claim 1, wherein the food item influencers comprise an influencer type, a food item or food item category, and spatiotemporal influence selectors for at least one of a date, a day, a meal, and combinations thereof.

4. The method of claim 1, wherein the brand influencers comprises a particular food item influencer.

5. The method of claim 1, wherein each subset of the second set of food item displays comprises a food item alteration UI activator, a food item removal control, and an item swap UI activator for each food item.

6. The method of claim 4 comprises:
receiving an input for the item swap UI activator of a particular food item;
displaying an item swap UI for the particular food item, the item swap UI comprising a food item nutrient comparison display including nutritional values of the particular food item in comparison to a candidate replacement food item, the candidate replacement food item generated by inputs received to a food item type filter, a food item quantity selector, a food item selector display, and a food item alteration control activator; and
replacing the particular food item from the subset of the second set of food items, with the candidate replacement food item, in response to receiving an input to the food item alteration control activator.

7. The method of claim 1, wherein the daily menu UI comprises a nutrition intake display displaying a second set of nutritional values configured by inputs from a menu view selector, and at least one nutrient subcategory display each comprising a list of nutrient items including nutrient item selectors.

8. The method of claim 6, wherein the daily menu UI displays an enhanced nutrient item display comprising a third set of nutritional values configured by at least one nutrient item selector and the menu view selector.

9. The method of claim 1, wherein displaying the shopping list configuration UI comprises:
displaying a shopping list configurator UI in response to an input to the list control activator; and
displaying a temporal subset of the second set of food item displays with corresponding temporally adjusted food item quantities in response to receiving temporal parameters through the shopping list configurator UI.

10. The method of claim 1, wherein the food log configuration UI comprises a food log nutritional information display, and a set of nutritional values configured by modifications to the food items in the food log subset of the second set of food item displays.

11. The method of claim 1 comprises: displaying a recipe creator UI comprising at least one of recipe description inputs, a recipe alteration activator, food item configurators, a visibility control selector, recipe instruction input and combinations thereof.

12. A system comprising: a user interface displayed on a machine display, the user interface configured to:
receive an input at a control interface;
send a control signal associated with the control interface to a controller in response to the input;
receive a display control signal from the controller; and
alter the machine display in response to the display control signal; and the controller, the controller configured to:
receive the control signal from the user interface;
operate one or more filters to select:
a first set of food items from a proximal food distributor database; and
a menu rules control;
generate a menu generation control signal utilizing the first set of food items selected and the menu rules control;
send the menu generation control signal to a menu generator;
receive a menu control signal from the menu generator, the menu control signal associated with a second set of food items in the proximal food distributor database, wherein the second set of food items is a subset of the first set of food items; and
send the display control signal to the user interface.

13. The system of claim 12, wherein the controller alters the menu generation control signal to influence selecting the second set of food items from the first set of food items.

14. The system of claim 12, wherein the controller is further configured to send a purchase control signal to a food distributor portal, the purchase control signal comprising instructions to operate the food distributor portal to select the second set of food items.

15. The system of claim 12, wherein the display control signal alters the user interface to display the second set of food items, the user interface further comprising one or more controls to alter the second set of food items.

16. The system of claim 12, wherein the user interface displays a daily menu UI comprising subsets of the second set of food items in a second set of food item displays, wherein the daily menu UI is configured by inputs to a temporal UI selector and a nutritional information display displaying a first set of nutritional values of the subsets of the second set of food items.

17. The system of claim 12, wherein the user interface displays a shopping list configuration UI comprising an item modification activator, a list modification activator, a list control activator, a shopping list display, and a second set of food item display, wherein the second set of food item display comprises the second set of food items and quantities of the second set of food items.

18. The system of claim 12, wherein the user interface displays a food log configuration UI comprising a food log subset of the second set of food item displays comprise a subset alteration UI activator and food items a food item selector, a food item modification control UI activator, and a food item removal control, wherein the second set of food item displays being configured by a temporal UI selector and each food item comprising a food item quantity.

19. The system of claim 12, where in the user interface displays a recipe configuration UI comprising a generated recipe list display, the generated recipe list display comprising a recipe descriptor, a recipe modification activator, a recipe removal control, and a recipe creator UI activator.

20. A method comprising:
receiving control signals from a user interface;
operating one or more filters to select:
a first set of food items from a proximal food distributor database; and
a menu rules control;
generating a menu generation control signal utilizing the first set of food items selected and the menu rules control;
sending the menu generation control signal to a menu generator, the menu generation control signal comprising instructions for the menu generator to generate a menu control signal, the menu control signal associated with a second set of food items in the proximal food distributor database, wherein the second set of food items is a subset of the first set of food items;
receiving the menu control signal from the menu generator; and
sending a display control signal to the user interface to alter a machine state of the user interface to display the second set of food items in response to receiving the menu control signal.
